(19) United States
(12) Patent Application Publication
(10) Pub. No.: US 2022/0380426 A1
(43) Pub. Date: Dec. 1, 2022

(54) HOMOGENEOUS MUTEINS OF THE HUMAN IL-27 ALPHA-SUBUNIT

(71) Applicant: TECHNISCHE UNIVERSITAT MUNCHEN, Munchen (DE)

(72) Inventors: Matthias FEIGE, Munchen (DE); Stephanie MULLER, Munchen (DE)

(73) Assignee: TECHNISCHE UNIVERSITAT MU, Munchen (DE)

(21) Appl. No.: 17/755,942

(22) PCT Filed: Nov. 12, 2020
(86) PCT No.: PCT/EP2020/081877

§ 371 Date: May 12, 2022

(30) Foreign Application Priority Data
Nov. 12, 2019 (EP) 19208453.1

(51) Int. Cl. C07K 14/54; C12N 15/63

(52) U.S. Cl. CPC C07K 14/54; C12N 15/63

(57) ABSTRACT

The present invention refers to a mutein of the α-subunit of human Interleukin 27 and of human heterodimeric Interleukin 27 according to the present invention. The present invention further refers to a nucleic acid molecule comprising a nucleotide sequence encoding a mutein of the α-subunit of human Interleukin 27 or of the human heterodimeric Interleukin 27. The invention further refers to a host cell containing a nucleic acid molecule comprising a nucleotide sequence encoding a mutein of the α-subunit of human Interleukin 27 or of the human heterodimeric Interleukin 27. The invention also refers to an immune modulator comprising a mutein of the α-subunit of human Interleukin 27 or of the human heterodimeric Interleukin 27, to the respective use thereof as well as to a method of producing a said muteins.

### CROSS-REFERENCE TO RELATED APPLICATIONS

The present application claims the benefit of priority of EP Patent Application No. 19 208 453.1 filed 12 Nov. 2019, the content of which is hereby incorporated by reference in its entirety for all purposes.

### FIELD OF THE INVENTION

The present invention refers to a mutein (mutant proteins) of the alpha-subunit of human Interleukin 27. Further, the present invention refers to a mutein of human heterodimeric Interleukin 27, wherein the alpha-subunit thereof is a mutein of the alpha-subunit of human Interleukin 27 as described herein. The invention also refers to a mutein of the alpha-subunit of human Interleukin 27, wherein the mutein comprises at least 60% sequence identity to the alpha-subunit of human Interleukin 27. The invention also refers to a mutein of human heterodimeric Interleukin 27, wherein the alpha-subunit thereof comprises at least 60% sequence identity to the alpha-subunit of human Interleukin 27. Interleukin 27 comprises the alpha-subunit p28 and the beta-subunit EBI3. The invention further refers to a nucleic acid molecule comprising a nucleotide sequence encoding a mutein of the alpha-subunit of human Interleukin 27 or encoding a mutein of the human heterodimeric Interleukin 27, wherein the alpha-subunit thereof is a mutein of the alpha-subunit of human Interleukin 27 as described herein. The invention further refers to a host cell containing a nucleic acid molecule comprising a nucleotide sequence encoding a mutein of the alpha-subunit of human Interleukin 27 or a mutein of the human heterodimeric Interleukin 27, wherein the alpha-subunit thereof is a mutein of the alpha-subunit of human Interleukin 27 as described herein. The invention also refers to an immune modulator comprising a mutein of the alpha-subunit of human Interleukin 27 or a mutein of the human heterodimeric Interleukin 27, wherein the alpha-subunit thereof is a mutein of the alpha-subunit of human Interleukin 27 as described herein. The present invention further refers to the use of a mutein of the alpha-subunit of human Interleukin 27 as described herein or a mutein of the human heterodimeric Interleukin 27 as described herein for the manufacture of a medicament, to a method of treating an Interleukin 27-mediated disease comprising the step of administering a composition comprising a mutein of the present invention to a mammal in need thereof as well as to a method of producing a mutein of the alpha-subunit of human Interleukin 27 or a mutein of the human heterodimeric Interleukin 27, wherein the alpha-subunit thereof is a mutein of the alpha-subunit of human Interleukin 27 as described herein or a mutein comprising at least 60% sequence identity to the alpha-subunit of human Interleukin 27.

### BACKGROUND OF THE INVENTION

A central tenet of the human immune system is the balanced regulation of pro- and anti-inflammatory responses. This allows rapid eradication of threats while protecting the host. Interleukins (ILs) are structurally diverse small secreted proteins that mediate pro- and anti-inflammatory responses to maintain this balance. Among those, the Interleukin 12 (IL-12) family, which comprises four established members (IL-12, IL-23, IL-27 and IL-35)′ (see FIG. 1), epitomizes this concept of balanced immune regulation: IL-12 and IL-23 are mostly pro-inflammatory cytokines, whereas IL-35 performs immune-suppressive roles^{1,2}. IL-27 is functionally diverse with immunomodulatory pro- and anti-inflammatory functions, acting on different types of T cells³. It can promote pro-inflammatory responses and synergize with IL-12 to induce interferon γ (IFNγ) production by naïve T cells and natural killer (NK) cells⁴; but IL-27 can also dampen immune responses by inducing IL-10 as an anti-inflammatory cytokine⁵⁻⁷ or inhibiting responses of T_{H}17 cells^{8,9}, a cell type that has come into focus due to its role in a large variety of immune-mediated human diseases¹⁰.

Interleukin 12 (IL-12) cytokines regulate T cell function and development, decisively influencing pro- versus anti-inflammatory responses. Each family member is a heterodimer, and additionally their isolated subunits regulate immune reactions^{11,12,24.} This endows the IL-12 family with unparalleled regulatory capacities but also puts high demands on their biosynthesis.

Features shared by the IL-12 family, however, go beyond this central role in connecting innate and adaptive immunity. All IL-12 cytokines show structural hallmarks that set this family apart from other interleukins: each of the IL-12 family members is a heterodimer composed of a 4-helical bundle α-subunit (IL-12α/p35, IL-23α/p19 and IL-27α/p28, respectively) and of a β-subunit composed of two fibronectin (Fn) domains (EBI3) or two Fn and one immunoglobulin (Ig) domains (IL-12β/p40)^{11,12}. Of note, despite their distinct roles in regulating immune responses, all heterodimeric IL-12 family members are made up of only these three α- and two β-subunits and even further members may exist¹³. IL-12β is shared by the pro-inflammatory family members IL-12 and IL-23 and EBI3 is shared by the immunomodulatory/anti-inflammatory members IL-27 and IL-35. This raises important questions about structural features that mediate assembly specificity versus promiscuity within this family. It also poses an extra demand on the machinery of protein folding and quality control in the endoplasmic reticulum (ER), where all IL-12 family members are assembled prior to secretion. Insights into IL-12 family cytokine folding and assembly are very limited so far. It has been shown that all human α-subunits are retained in cells in isolation and depend on assembly with their cognate β-subunit in order to be secreted^{4,14,15}. In the case of the family's founding member, IL-12, assembly-induced folding of the IL-12α-subunit by IL-12β underlies these processes¹⁶, but otherwise the underlying mechanisms remain ill-defined.

Concerning the present invention, the inventors have focused on the structurally ill-characterized yet functionally highly diverse family member IL-27 (see FIG. 2A). In this regard it has already been found that in contrast to its human orthologue, the mouse IL-27 alpha-subunit (p28) can be secreted in isolation without its beta-subunit, whereas the secretion of human IL-27α strictly depends from EBI3⁴ (see FIGS. 2B and C). The different secretion behavior of human and murine IL-27 alpha could be attributed to the fact that murine IL-27 alpha can form a disulfide bridge, which stabilizes the protein, whereas human IL-27 alpha is unable to do so and therefore requires EBI3 for secretion. If a second cysteine is inserted into human IL-27 alpha by mutagenesis, human IL-27 alpha can also form a disulfide bond and be secreted autonomously (see FIG. 2D). Furthermore, an immuno-inhibitory/modulatory effect has been described for murine IL-27 alpha, also referred to as IL-30^{17,20}. It has also been shown that even human IL-27 alpha capable of autonomous secretion has immunomodulatory activity. However, its activity in STAT phosphorylation assays is 700-fold weaker than that of the heterodimer IL-27²¹.

Accordingly, there is a need in the art to improve the immunoinhibitory/-modulatory activity of a human α-subunit of human IL-27 or even of human heterodimer IL-27. The technical problem underlying the present application is thus to comply with these needs.

### SUMMARY OF THE INVENTION

The present inventors have developed a mutein of the alpha-subunit of human Interleukin 27, which is modified by specific point mutations or by particular deletions, which is more homogeneous while maintaining its protein functionality and in some cases even has an improved activity in comparison to the unmutated, heterogeneous human alpha subunit of human Interleukin 27. The inventors surprisingly found that a targeted, rational modification of the alpha subunit of human IL-27 by single point mutations, and also particular deletions of the alpha subunit of human IL-27 results in the prevention of O-glycosylations of said protein which is important for its homogeneity while maintaining its functionality and in some cases even enhanced activity compared to unmutated, heterogeneous human alpha subunit of human Interleukin 27.

Accordingly, in a first aspect, the present invention relates to a mutein of the α-subunit of human Interleukin 27, wherein at least one of the amino acid residues of the α-subunit of human Interleukin 27 selected from the group consisting of sequence positions 187, 238 and 240 is/are mutated. The invention also provides a mutein of the α-subunit of human Interleukin 27, wherein the mutein comprises at least 60% sequence identity to the α-subunit of human Interleukin 27 as defined herein.

In yet another aspect, the invention may also provide a mutein of the α-subunit of human Interleukin 27, wherein the residue at amino acid position 234 is mutated. The invention may also comprise a mutein of the α-subunit of human Interleukin 27, wherein the residue at amino acid position 238 is mutated. Preferably, the invention encompasses a mutein of the α-subunit of human Interleukin 27, wherein the residues at amino acid positions 234 and 238 are mutated.

In a second aspect, the present invention provides a mutein of human Interleukin 27, comprising an α-subunit p28 and a β-subunit EBI3, wherein the α-subunit is a mutein of the α-subunit of human Interleukin 27 as described herein. In a further embodiment thereof, the α-subunit is a mutein comprising at least 60% sequence identity to the α-subunit of human Interleukin 27 as described herein.

In a third aspect, the present invention provides a nucleic acid molecule comprising a nucleotide sequence encoding a mutein of human Interleukin 27 or a mutein of the α-subunit of human Interleukin 27 according to the present invention. In a further embodiment thereof, the nucleic acid molecule comprises a nucleotide sequence encoding a mutein of the α-subunit of human Interleukin 27, wherein the mutein comprises at least 60% sequence identity to the α-subunit of human Interleukin 27.

In a fourth aspect, the present invention provides also a host cell containing a nucleic acid molecule according to the present invention.

In a fifth aspect, the present invention provides an immune modulator comprising a mutein according to the present invention.

In a sixth aspect, the present invention provides the use of a mutein according to the present invention for the manufacture of a medicament for treating an infectious disease, an autoimmune disease, cancer, multiple sclerosis, a transplantation-related disease, such as Graft-versus-Host-disease, a chronic inflammatory disease, such as chronic inflammatory bowel disease, acute inflammatory disease, sepsis, septic shock, diabetes or asthma in a mammal.

In a related aspect, the present invention provides a mutein according to the present invention for use in therapy. Further, the present invention provides a mutein according to the present invention for use in the treatment of an infectious disease, an autoimmune disease, cancer, multiple sclerosis, a transplantation-related disease, such as Graft-versus-Host-disease, a chronic inflammatory disease, such as chronic inflammatory bowel disease, acute inflammatory disease, sepsis, septic shock, diabetes or asthma.

The present invention also provides a method of treating an Interleukin 27-mediated disease, preferably an infectious disease, an autoimmune disease, cancer, multiple sclerosis, a transplantation-related disease, such as Graft-versus-Host-disease, a chronic inflammatory disease, such as chronic inflammatory bowel disease, acute inflammatory disease, sepsis, septic shock, diabetes or asthma in a mammal, comprising the step of administering a composition comprising a mutein as described herein to a mammal in need thereof.

Additionally, the present invention provides a method of producing a mutein as described herein, comprising the steps of:

introducing into a nucleic acid molecule encoding the human Interleukin 27 polypeptide or the human Interleukin 27 α-subunit polypeptide or the polypeptide comprising at least 60% sequence identity to the human Interleukin 27 α-subunit polypeptide a nucleotide sequence mutating at least one amino acid residues of human Interleukin 27 or of the α-subunit of human Interleukin 27 or of the polypeptide comprising at least 60% sequence identity to the human Interleukin 27 α-subunit polypeptide selected from the group consisting of sequence positions 187, 238 and 240, and
introducing the obtained nucleic acid molecule for expression into a suitable host cell or into a suitable cell extract or cell lysate.

These aspects of the invention will be more fully understood in view of the following drawings, detailed description and non-limiting examples.

### BRIEF DESCRIPTION OF THE DRAWINGS

The accompanying drawings are included to further an understanding of the embodiments that are incorporated in and constitute a part of this specification. The drawings illustrate embodiments and together with the description serve to explain principles of embodiments. Other embodiments and many of the intended advantages of embodiments will be readily appreciated, as they become better understood by reference to the detailed description. The elements of the drawings are not necessarily to scale relative to each other.

FIG. 1 shows the schematic representation of IL-12 family members, their structure and immunological activity.

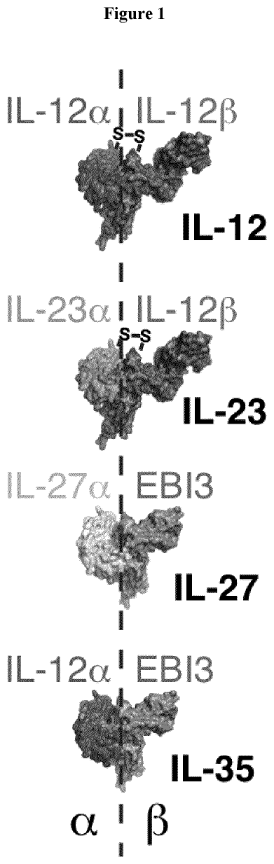

FIG. 2(a) shows the schematic representation of the IL-27 structure. FIG. 2(b) shows the secretion competency of the IL-27 subunits in humans and FIG. 2(c) shows the secretion competency of the IL-27 subunits in mice. FIG. 2(d) shows the engineered autonomously secreting human IL-27alpha L162C subunit (SEQ ID NO: 1).

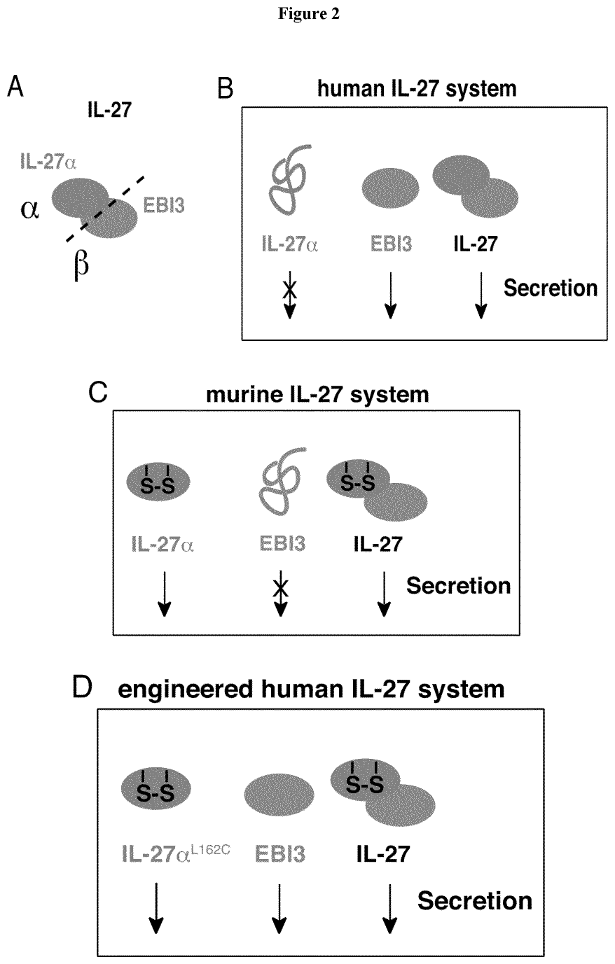

FIG. 3 shows the analysis of glycosylation status of human IL-27alpha. Secreted IL-27alpha shows two species resulting from glycolysis (in (a) and (b)). FIG. 3(a) shows that secreted V5-tagged IL-27alpha was treated with N-glycosidases (E: EndoH and P: PNGaseF) and O-glycosidase (O). The analysis shows that IL-27alpha is not N-glycosylated but O-glycosylated. The protein runs faster after cleavage of the sugar residues. FIG. 3(a) shows that untagged IL-27alpha is also O-glycolysed.

FIG. 4(a) shows analysis of the N-glycosylation status of murine IL-27alpha (SEQ ID NO: 10). Secreted murine IL-27alpha was treated with N-glycosidase PNGaseF (+). The analysis shows that IL-27alpha is N-glycosylated as the protein runs faster after cleavage of sugar residues by PNGases. FIG. 4(b) shows O-glycosidase analysis and that murine IL-27alpha is not O-glycosylated.

FIG. 5 shows sequence comparison of human IL-27alpha^{L162C} (O-glycosyated) (SEQ ID NO: 1) and murine IL-27alpha (N-glycosylated) (SEQ ID NO: 10). Threonines and serines, which are present in humans but not in mice, are printed in italics. Threonines and serines which are exposed in the structure prediction of the human protein are additionally marked by an arrow.

FIG. 6 shows the structural model of human IL-27alpha^{L162C} (SEQ ID NO: 1). Threonines and serines contained in humans but not in mice are shown as Ser110, Ser202, Ser187, Thr238 and Ser240. Threonines and serines that are solvent exposed are additionally marked by an arrow.

FIG. 7 shows mutation of potential O-glycosylation sites in human IL-27alpha^{L162C}. Threonines and serines that could potentially be O-glycosylated were mutated to alanines. The mutant IL-27alpha^{L162C,T238A,S240A} (SEQ ID NO: 8) shows only one protein species which migrates faster than the other mutants due to its lower molecular weight.

FIG. 8 shows that IL-27alpha^{L162C,T238A,S240A} (SEQ ID NO: 8) is not O-glycosylated. Secreted IL-27alpha^{L162C,T238A,S240A} (SEQ NO: 8) has been treated with O-glycosidase and does not migrate faster than the negative control after treatment. This shows that IL-27alpha^{L162C,T238A,S240A} (SEQ ID NO: 8) is not O-glycosylated.

FIG. 9 shows the expression of IL-27alpha^{L162C,T238A,S240A} (SEQ ID NO: 8) in mammalian cells with a concentration of 4.1 μg/mL.

FIG. 10 shows that unglycosylated IL-27alpha^{L162C,T238A,S240A} (SEQ ID NO: 8) is 8.5 times more active than O glycosylated IL-27alpha^{L162C} (SEQ ID NO: 1). BL-2 cells expressing the IL-27 receptor were incubated for 60 minutes with 1000 ng/mL IL-27alpha^{L162C} (SEQ ID NO: 1) or IL-27alpha^{L162C,T238A,S240A} (SEQ ID NO: 8) and STAT1 activation was determined by immunoblotting against phosphorylated STAT1.

FIG. 11(a) shows the structural model of hIL-27alpha^{L162C} (SEQ ID NO: 1), whereas Leu234, Thr238 and Ser240 are shown therein. FIG. 11(b) shows the amino acid sequence of hIL-27alpha^{L162C} (SEQ ID NO: 1). Leu234, Thr238 and Ser240 are marked with arrows. The structural model does not include the ER signal sequence which is underlined in the amino acid sequence.

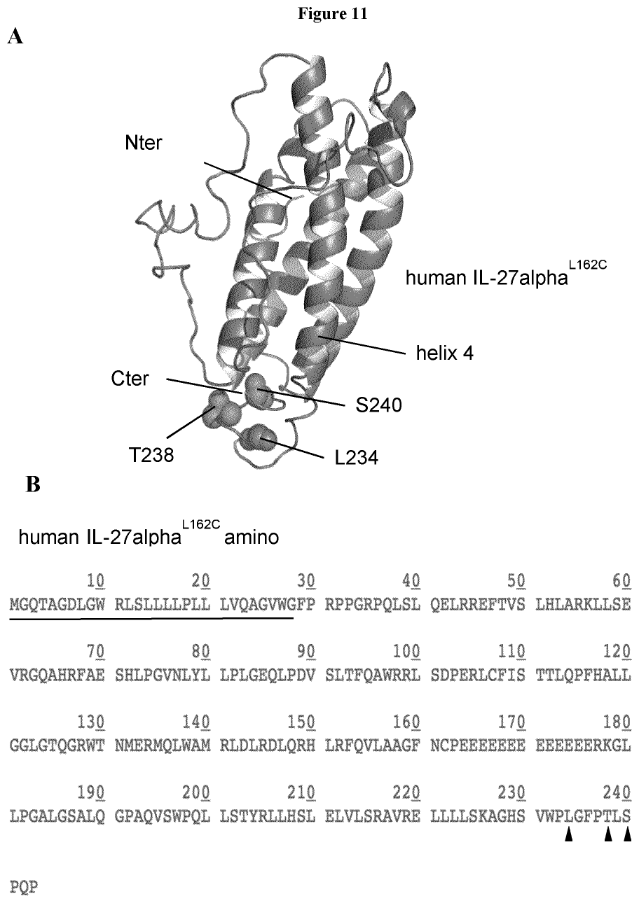

FIG. 12 shows different IL-27α pairs (without comprising the substitution of leucine 162 with cysteine (L162C)) being tested for the functionality on BL-2 cells. These either consisted of the WT pair (being O-/N-glycosylated), only hIL-27α lacking O-glycosylation, only hEBI3 lacking N-glycosylation, or the pair of both subunits lacking O-/N-glycosylation (hIL-27alpha^{T238A,S240A (=ΔO)} and hEBI3^{N55QN105Q (=ΔN)}). These data show that O-glycosylation is not necessary for the function of heterodimeric IL-27.

FIG. 13 shows IL-27α (comprising the substitution of leucine 162 with cysteine (L162C)) was truncated after Gly228 to delete its C-terminal O-glycosylation sites. It shows that the C-terminus is dispensable for IL-27α (L162C) functions.

### DETAILED DESCRIPTION

The following language and descriptions of certain preferred embodiments of the present invention are provided in order for further understanding of the principles of the present invention. However, it will be understood that no limitations of the present invention are intended, and that further alterations, modifications, and applications of the principles of the present invention are also included.

The present invention is directed to a mutein of the α-subunit of human Interleukin 27 (IL-27), wherein at least one of the amino acid residues of the α-subunit of human IL-27 selected from the group consisting of sequence positions 187, 238 and 240 is/are mutated.

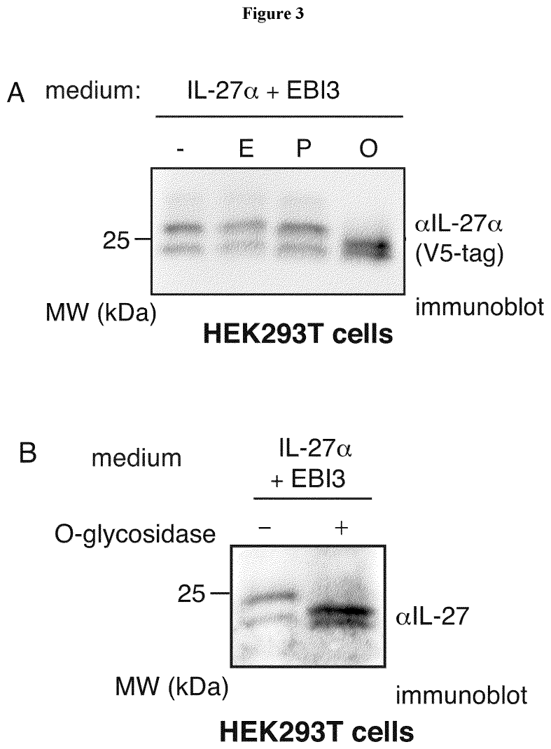

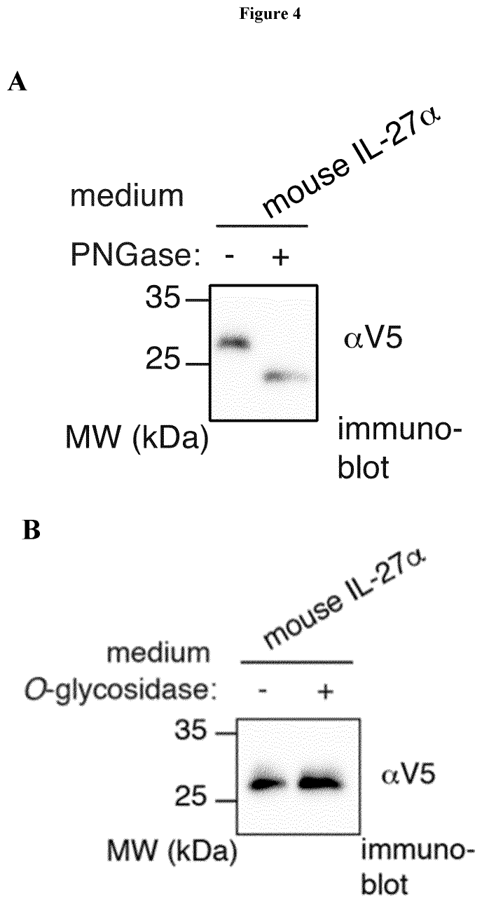

Secretory proteins, such as interleukins, are often glycosylated. This modification includes N-glycosylations on asparagine residues²² or, often more heterogeneous, 0-glycosylations on serine and threonine residues²³. Human IL-27 alpha is however not N-glycosylated but O-glycosylated (see FIG. 3). Murine IL-27 alpha, on the other hand, is N-glycosylated but not O-glycosylated (see FIG. 4).

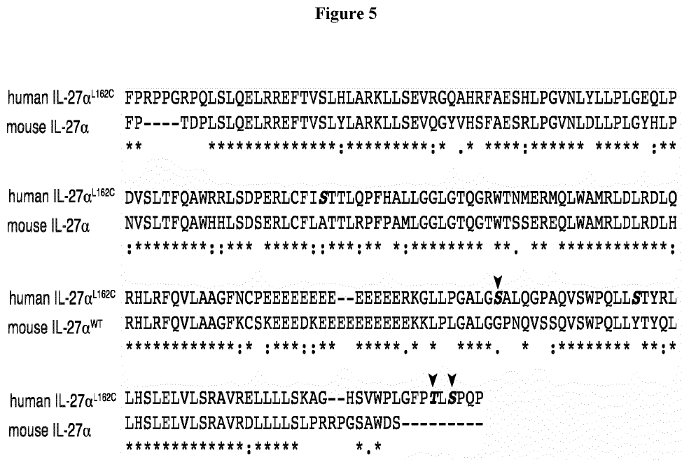

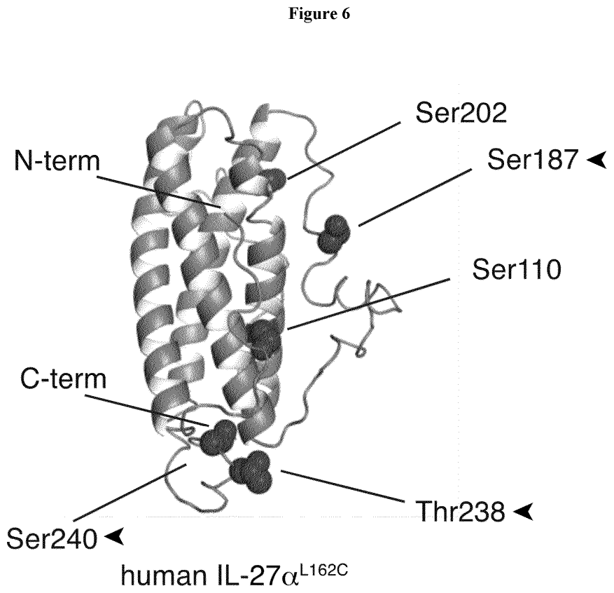

IL-27 alpha is a secretory protein that is folded in the endoplasmic reticulum (ER) and O-glycosylated on the way to the extracellular space in the Golgi compartment. The formation of a three-dimensional protein structure usually takes place in the ER before a protein reaches the Golgi apparatus. O-glycosylation in the Golgi apparatus is therefore only possible on surface-exposed serine and threonine residues. By performing a sequence and structure alignment of murine and human Interleukin 27, the inventors have identified surface-exposed serine and threonine residues which are responsible for O-glycosylations in the alpha subunit of human Interleukin 27 (see FIGS. 5 and 6).

According to the present invention, the inventors have then exchanged the detected serine and threonine residues for an alanine. By replacing the surface-exposed serine and threonine residues of the protein by alanine residues, the O-glycosylations of the protein in the Golgi apparatus is prevented. In particular, this analysis revealed that the mutation of specific amino acid residues of the α-subunit of human IL-27 selected from the group consisting of sequence positions 187, 238 and 240 results in the prevention of O-glycosylations at at least one of said mutated amino acid residues, which results in a more homogeneous protein while maintaining complete functionality of said protein. The same effect is also achieved when specific deletions of the alpha subunit of human IL-27 as described elsewhere herein have been performed.

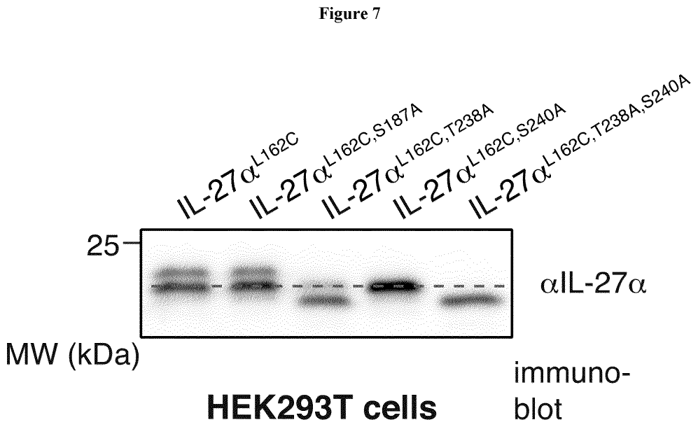

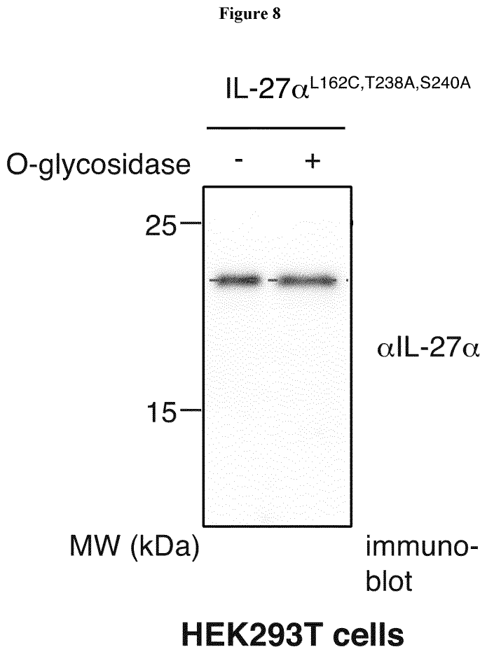

Comparisons with mobility on SDS-PAGE gels of the secretory-competent protein (IL-27 alpha^{L162C}) show that the exchange of threonine 238 (Thr 238) and serine 240 (Ser 240) for alanine residues results in a single, faster migrating species on the immunoblot (see FIG. 7). This is indicative of a lower molecular weight due to the lack of O-glycosylation. Treatment with O-glycosidase shows that the mutant in which Thr238 and Ser240 are substituted for alanine has no O-glycosylation (see FIG. 8).

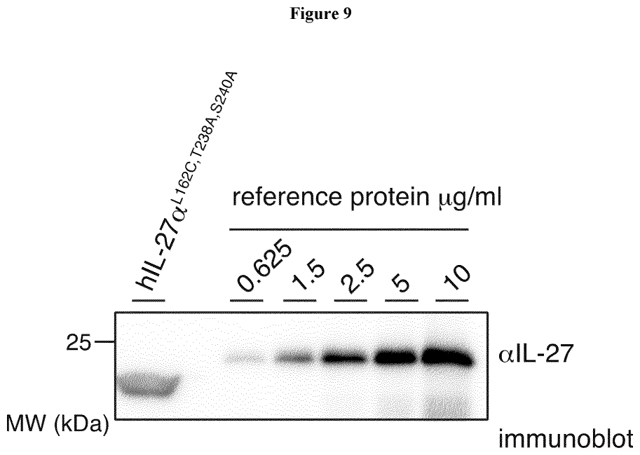

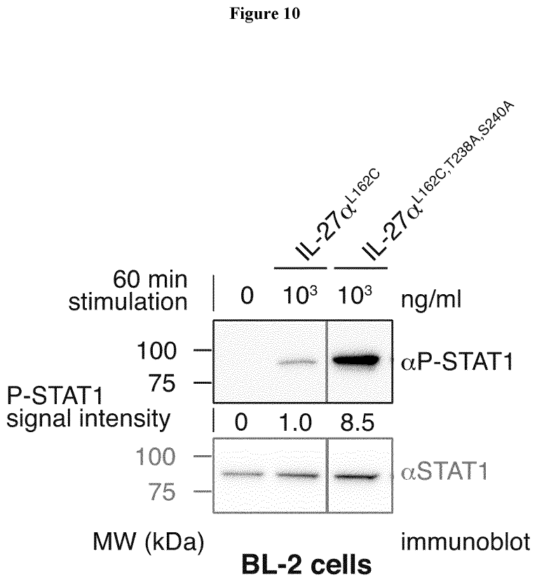

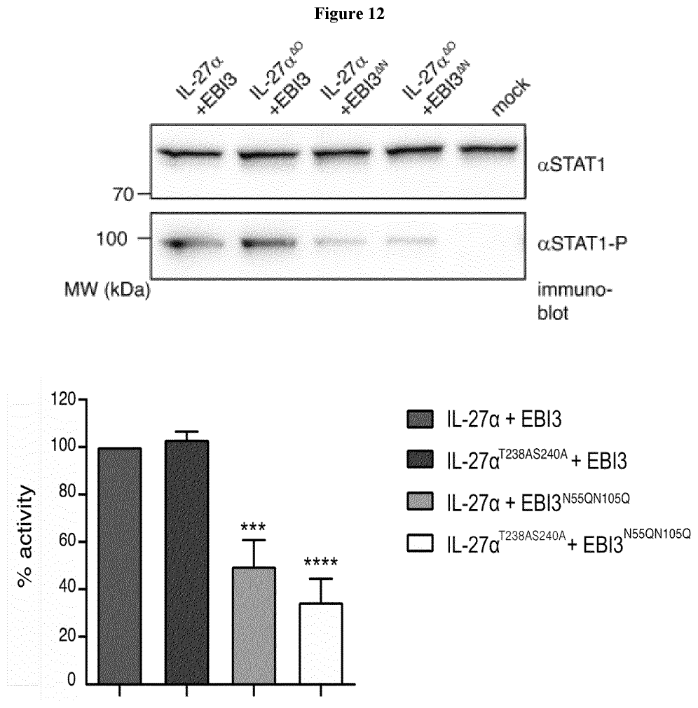

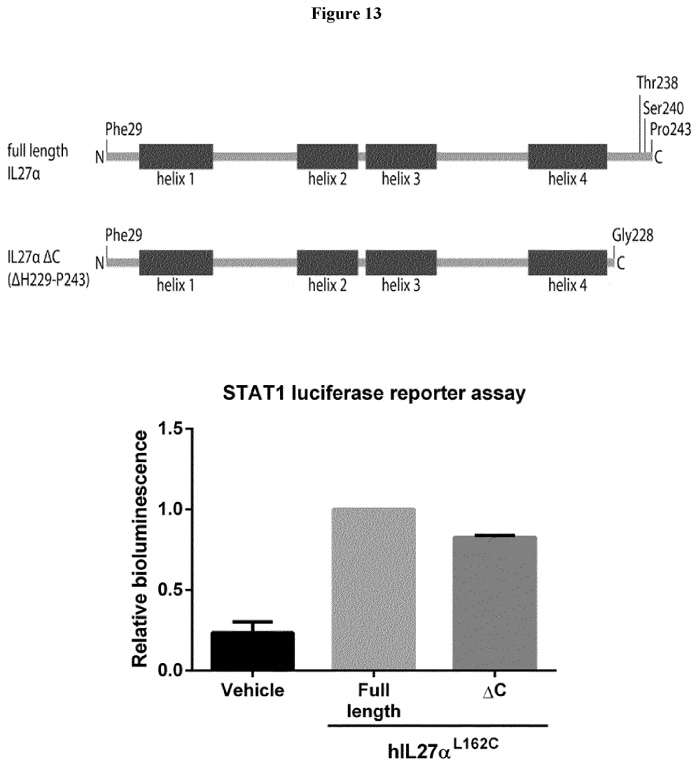

Since O-glycans are known to be very heterogeneous, the protein according to the present invention without glycosylations is very homogeneous. This is particularly very beneficial with regard to a possible medical use and the resulting requirements for a biopharmaceutical. The new, homogeneous protein without O-glycosylation was subsequently produced in a mammalian cell system (see FIG. 9) and tested for its activity in an immune cell assay. The homogeneous alpha subunit of human Interleukin 27 without O-glycosylations has additionally an about 10-fold higher activity as the unmutated, heterogeneous alpha subunit of human Interleukin 27 in said activity immune cell assay a (see FIG. 10). Further, the data also show that O-glycosylation in the alpha subunit of human Interleukin 27 without comprising the substitution of leucine 162 with cysteine (L162C) has no effect on the function of heterodimeric IL-27. Thus, if at least one of the amino acid residues of the α-subunit of human Interleukin 27 selected from the group consisting of sequence positions 238 and 240 is/are mutated, more homogeneous IL-27 is obtained while maintaining complete functionality (see FIG. 12). It has also been demonstrated that even the deletion of the complete C-terminus in human IL-27alpha does not impair its functionality—while at the same time creating a homogeneous species (see FIG. 13).

Thanks to its homogeneity without the loss of functionality, it may be suitable as a new immunomodulator, for example in sepsis therapy. The suitability as a new immunomodulator may also be further improved by its additional enhanced activity in comparison to the wild type being used. It also results in providing lower doses of human Interleukin 27 alpha as biopharmaceutical or even of heterodimeric Interleukin 27 as a biopharmaceutical containing Interleukin 27 alpha, which even makes the production of such biopharmaceuticals more cost-effective. Thus, for obtaining a homogeneous product in the development of human IL-27 alpha-subunit as a biopharmaceutical, and also in the development of heterodimeric IL-27 as a biopharmaceutical containing IL-27 alpha-subunit, the removal of O-glycosylation sites has been proven to be beneficial.

Lastly, due to the present invention, it is possible to design an autonomously folding human IL-27 alpha-subunit, which is homogeneous and acts as an improved immune modulator. In this context, it is noted that the term “human Interleukin 27 (IL-27) alpha-subunit” or “human Interleukin 27 (IL-27) α-subunit” as used herein refers inter alia to the polypeptide sequence of SEQ ID NO: 1 that has been deposited under UniProtKB accession number Q8NEV9. The term “human Interleukin 27 (IL-27) beta-subunit” or “hEBI3” (SEQ ID NO: 9) as used herein refers to the polypeptide sequence deposited under UniProtKB accession number Q14213 that associates with the human IL-27 alpha-subunit to form the Interleukin 27, a heterodimeric cytokine which functions in immune repsonses. The term “mouse Interleukin 27 (IL-27) alpha-subunit” or “mIL-27□” (SEQ ID NO: 10) as used herein refers to the polypeptide sequence deposited under genbank identifier NP 663611.1. The term “mouse Interleukin 27 (IL-27) beta-subunit” or “mEBI3” as used herein refers to the polypeptide sequence deposited under UniProtKB accession number O35228. The term “human Interleukin 27 (IL-27) beta-subunit” or “hEBI3” as used herein refers to the polypeptide sequence deposited under UniProtKB accession number Q14213 (SEQ ID NO: 12). Accordingly, the term “IL-27” or “Interleukin 27” refers to the heterodimeric cytokine formed by the IL-27 alpha- and IL-27 beta-subunit. The beta-subunit EBI3 is however not O-glycosylated, but N-glycosylated. The EBI3 may be N-glycosylated at multiple sites. The hEBI3 may be N-glycosylated at at least one of the amino acid residues selected from the group consisting of sequence positions 55 and 105 corresponding to sequence position of SEQ ID NO: 12. When at least one of the amino acid residues of the human beta-subunit EBI3 of human Interleukin 27 at sequence positions 55 and 105 corresponding to sequence position of SEQ ID NO: 12 is mutated, it refers to the hEBI3 mutant lacking N-glycosylation. In this context, the mutation refers to replacing the at least one of the amino acid residues of the beta-subunit EBI3 of human Interleukin 27 at sequence positions 55 and 105 corresponding to sequence position of SEQ ID NO: 12 by glutamine (Gln/Q).

When the term “human Interleukin 27 (IL-27) alpha-subunit” or “human Interleukin 27 (IL-27) α-subunit” as used herein refers to the polypeptide sequence of SEQ ID NO: 1, SEQ ID NO: 1 either refers as described elsewhere herein to the wild type (WT) human Interleukin 27 (IL-27) alpha-subunit/human Interleukin 27 (IL-27) α-subunit comprising at position 162 leucin (Uniprot Accession Number Q8NEV9) or to the mutant of the WT human Interleukin 27 (IL-27) alpha-subunit/human Interleukin 27 (IL-27) α-subunit comprising the substitution of leucine 162 with cysteine (L162C). The latter is preferred in the present invention. The mutant of the present invention, namely the mutein of the α-subunit of human Interleukin 27 as defined elsewhere herein is derived from the WT human Interleukin 27 (IL-27) α-subunit having SEQ ID NO: 1 as it is defined by the present invention. When the term “human Interleukin 27 (IL-27) alpha-subunit” or “human Interleukin 27 (IL-27) α-subunit” as used herein refers to the polypeptide sequence of SEQ ID NO: 1 comprising the substitution of leucine 162 with cysteine (L162C), said mutein thereof is secretion-competent. Such term may also refer to the polypeptide sequence comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the α-subunit of human Interleukin 27, wherein the amino acid residue at sequence position 162 (leucine) corresponding to the sequence position of SEQ ID NO: 1 is replaced by cysteine (L162C) compared to the α-subunit of human Interleukin 27 and then said mutein is still secretion-competent. Accordingly, a mutein of human Interleukin 27, comprising an α-subunit p28 and a β-subunit EBI3, wherein the α-subunit is a mutein of the α-subunit of human Interleukin 27 which refers to SEQ ID NO: 1 having the substitution of leucine 162 with cysteine (L162C), said mutein thereof is secretion-competent. The disclosure referring to the sequence identity above and that said mutein is still secretion-competent is therefore also applicable to the mutein of human Interleukin 27, comprising an α-subunit p28 and a β-subunit EBI3. Accordingly, when there is disclosure to SEQ ID NOs: 2-8 in the present invention, also these particular sequences are based on SEQ ID NO: 1 having either at position 162 leucin or comprising the substitution of leucine 162 with cysteine (L162C). In some cases, the substitution of leucine 162 with cysteine (L162C) may be preferred for SEQ ID NOs: 2-8.

The term “secreting” or “secretion” is used in the present invention in its regular meaning to mean the active export of a protein from a (eukaryotic such as a human) cell into the extracellular environment. Generally secretion occurs through a secretory pathway in the cell, for example, in eukaryotic cells, this involves the endoplasmic reticulum and the golgi apparatus.

A mutein according to the present invention is “secretion-competent” or “comprises secretion competence”, when the mutein is able to perform a complete passage through the secretory pathway of the cell and through the cytoplasmic membrane.

In contrast thereto, the term “non-secretion competent” muteins means in the context of the present invention muteins, which are not naturally secreted from the cell into the extracellular environment.

In accordance with the above disclosure, in the mutein of the α-subunit of human IL-27 of the present invention at least one of the amino acid residues at sequence positions 187, 238 and 240 of SEQ ID NO. 1 can be mutated. This means, a mutein of the present invention can comprise a single mutation at one of these sequence positions, but also a mutation at two or all three of these sequence positions. In this context and as used in the present invention, the term “mutated” refers to a replacement/substitution by another amino acid such as by single point mutations as defined elsewhere herein or it refers to a deletion of particular amino acids as defined elsewhere herein. Any additional mutation(s) in SEQ ID NO: 1 not explicitly disclosed herein, which however does not affect the protein functionality of the α-subunit of human IL-27 or of human heterodimeric Interleukin 27 may also be comprised herein by the term “mutated”. Also, where applicable, any additional mutation(s) in SEQ ID NO: 1 not explicitly disclosed herein, which does not impair secretion-competence may also be comprised herein by the term “mutated”. Whenever the term “mutated” is used, the term “replaced” may be used interchangeably. In some instances, the term “deleted” may be used interchangeably with the term “mutated”. This is also applicable vice versa.

In more detail, the term “mutation” as used herein means that the experimental conditions are chosen such that the amino acid naturally occurring at a given sequence position of the α-subunit of human IL-27 of the present invention can be substituted by at least one amino acid that is not present at this specific position in the respective natural polypeptide sequence. Thus the term “mutation” includes a substitution of at least one amino acid not present at this specific position in the respective natural polypeptide sequence. The term “mutation” also includes the (additional) modification of the length of sequence segments by deletion or insertion of one or more amino acids. For example, one amino acid at a chosen sequence position can also be replaced by a stretch of two, three or more random mutations, leading to an insertion of one, two or more amino acid residues compared to the length of the respective segment of the wild type protein. Said term also includes an inversion, which refers to a kind of mutation in which the order of the amino acids in a section of the amino acid sequence is reversed with respect to the remainder of the amino acid sequence. Thus, any types and numbers of mutations, including substitutions, deletions, and insertions, are envisaged as long as a provided mutein retains its functionality/secretion competence, where applicable of the α-subunit of human IL-27 or of human heterodimeric Interleukin 27, and/or it has a sequence identity that it is at least 60%, including at least 65%, at least 70%, at least 75%, at least 80%, at least 85% or higher identity to the amino acid sequence of SEQ ID NO.: 1 of the reference WT human Interleukin 27 (IL-27) α-subunit.

Thus, it is comprised by the present invention that in the mutein of the α-subunit of human IL-27 of the present invention, the amino acid residue at sequence position 187 corresponding to the sequence position of SEQ ID NO. 1 can be mutated as described herein. Also comprised herein is that in the mutein of the α-subunit of human IL-27 of the present invention the amino acid residue at sequence position 238 corresponding to the sequence position of SEQ ID NO. 1 can be mutated as described herein. In another embodiment comprised by the present invention in the mutein of the α-subunit of human IL-27 of the present invention the amino acid residue at sequence position 240 corresponding to the sequence position of SEQ ID NO. 1 can also be mutated as described herein.

In yet another embodiment comprised by the present invention, in the mutein of the α-subunit of human IL-27 of the present invention the amino acid residues at a sequence positions 187 and 238 corresponding to the sequence positions of SEQ ID NO. 1 can also be mutated as described herein. In yet another embodiment comprised by the present invention, in the mutein of the α-subunit of human IL-27 of the present invention the amino acid residues at a sequence positions 187 and 240 corresponding to the sequence positions of SEQ ID NO. 1 can also be mutated as described herein. Finally, in yet another embodiment comprised by the present invention, in the mutein of the α-subunit of human IL-27 of the present invention the amino acid residues at a sequence positions 187, 238 and 240 corresponding to the sequence positions of SEQ ID NO. 1 can also be mutated as described herein.

Preferably, in the mutein of the α-subunit of human IL-27 of the present invention the amino acid residues at a sequence positions 238 and 240 corresponding to the sequence position of SEQ ID NO. 1 are mutated as described herein.

The mutation can be any amino acid that cannot be O-glycosylated, thus any amino acid except serine or threonine.

In one embodiment of the present invention, in the mutein of the α-subunit of human IL-27 of the present invention, the amino acid residue at sequence position 187 corresponding to the sequence position of SEQ ID NO. 1 can be replaced by alanine (SEQ ID NO: 2). In another embodiment of the present invention, in the mutein of the α-subunit of human IL-27 of the present invention the amino acid residue at sequence position 238 corresponding to the sequence position of SEQ ID NO. 1 can be replaced by alanine (SEQ ID NO: 3). In another embodiment of the present invention, in the mutein of the α-subunit of human IL-27 of the present invention the amino acid residue at sequence position 240 corresponding to the sequence position of SEQ ID NO. 1 can be replaced by alanine (SEQ ID NO: 4).

In yet another embodiment of the present invention, in the mutein of the α-subunit of human IL-27 of the present invention the amino acid residues at sequence positions 187 and 238 corresponding to the sequence position of SEQ ID NO. 1 can be replaced by alanine (SEQ ID NO: 5). In yet another embodiment of the present invention, in the mutein of the α-subunit of human IL-27 of the present invention the amino acid residues at sequence positions 187 and 240 corresponding to the sequence position of SEQ ID NO. 1 can be replaced by alanine (SEQ ID NO: 6). In yet another embodiment of the present invention, in the mutein of the α-subunit of human IL-27 of the present invention the amino acid residues at sequence positions 187, 238 and 240 corresponding to the sequence position of SEQ ID NO. 1 can be replaced by alanine (SEQ ID NO: 7).

Most preferably, in the mutein of the α-subunit of human IL-27 of the present invention the amino acid residues at sequence positions 238 and 240 corresponding to the sequence position of SEQ ID NO. 1 are replaced by alanine (SEQ ID NO: 8).

In line with the above, it is within the scope of the present invention that the above mentioned mutations of the α-subunit of human IL-27 at the amino acid residues 187, 238 and 240 to alanine can form muteins with 1, 2, or 3 alanines at any of the mentioned positions 187, 238 and 240 corresponding to the sequence position of SEQ ID NO. 1 of the α-subunit of human IL-27.

In yet another embodiment of the present invention, in the mutein of the α-subunit of human IL-27 of the present invention the amino acid residue at at least one of sequence positions 187, 238 and 240 corresponding to the sequence position of SEQ ID NO. 1 can be replaced by any amino acid, which cannot be O-glycosylated, thus any amino acid except serine or threonine.

It is also thus possible that a mutein of the α-subunit of human IL-27 of the present invention can further comprise one or more disulfide-bridges. In addition or alternatively, the mutein of the α-subunit of human IL-27 of the present invention can further comprise one or more salt bridges that act as structural homologue of the intra-chain disulfide bridge formed, for example, between the naturally occurring cysteine residue present at sequence position 107 corresponding to the sequence position of SEQ ID NO. 1 of the α-subunit of human IL-27 and a cysteine residue introduced at position 162 corresponding to the sequence position of SEQ ID NO. 1 of the α-subunit of human IL-27. The salt bridge may, for example, arise from the anionic carboxylate (RCOO⁻) group of either aspartic acid or glutamic acid and the cationic ammonium (RNH₃⁺) from lysine or the guanidinium (RNHC(NH₂)₂⁺) of arginine. Although these are the most common, other residues with ionizable side chains such as histidine, tyrosine, and serine can also participate in the formation of a salt bridge.

In a further aspect, the present invention provides a mutein of the α-subunit of human Interleukin 27 comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1 of the α-subunit of human Interleukin 27, wherein in this mutein at least one of the amino acid residues selected from the group consisting of sequence positions 187, 238 and 240 corresponding to the sequence position of SEQ ID NO.: 1 is/are mutated compared to the α-subunit of human Interleukin 27. In this connection, it is noted that the 0-subunit of mouse Interleukin 27 (SEQ ID NO: 10) has an amino acid length of 234 residues, while the 0-subunit of human Interleukin 27 has an amino acid length of 243 residues. The sequence identity between the SEQ ID NO: 1 and the SEQ ID NO: 10 has been determined as being 75%. Thus, it is preferred that the mutein of the present invention comprises an amino acid sequence with at least 76% sequence identity to the amino acid sequence of SEQ ID NO.: 1 of the α-subunit of human Interleukin 27 having the defined mutations mentioned elsewhere herein. Thus, said preferred sequence identity of at least 76% such as 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1 of the α-subunit of human Interleukin 27 may be combined with each embodiment herein.

In another aspect, the present invention provides a mutein of the α-subunit of human Interleukin 27 comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1 of the α-subunit of human Interleukin 27, wherein in this mutein the amino acid residue at sequence position 187 corresponding to the sequence position of SEQ ID NO.: 1 is mutated, preferably replaced by alanine (SEQ ID NO: 2), compared to the α-subunit of human Interleukin 27.

In a more preferred aspect, the present invention provides a mutein of the α-subunit of human Interleukin 27 comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1 of the α-subunit of human Interleukin 27, wherein in this mutein the amino acid residue at sequence position 238 corresponding to the sequence position of SEQ ID NO.: 1 is mutated, preferably replaced by alanine (SEQ ID NO: 3), compared to the α-subunit of human Interleukin 27.

In another more preferred aspect, the present invention provides a mutein of the α-subunit of human Interleukin 27 comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1 of the α-subunit of human Interleukin 27, wherein in this mutein the amino acid residue at sequence position 240 corresponding to the sequence position of SEQ ID NO.: 1 is mutated, preferably replaced by alanine (SEQ ID NO: 4), compared to the α-subunit of human Interleukin 27.

In another aspect, the present invention provides a mutein of the α-subunit of human Interleukin 27 comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1 of the α-subunit of human Interleukin 27, wherein in this mutein the amino acid residues at sequence positions 187 and 238 corresponding to the sequence position of SEQ ID NO.: 1 are mutated, preferably replaced by alanine (SEQ ID NO: 5) compared to the α-subunit of human Interleukin 27.

In another aspect, the present invention provides a mutein of the α-subunit of human Interleukin 27 comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1 of the α-subunit of human Interleukin 27, wherein in this mutein the amino acid residues at sequence positions 187 and 240 corresponding to the sequence position of SEQ ID NO.: 1 are mutated, preferably replaced by alanine (SEQ ID NO: 6) compared to the α-subunit of human Interleukin 27.

In another aspect, the present invention provides a mutein of the α-subunit of human Interleukin 27 comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1 of the α-subunit of human Interleukin 27, wherein in this mutein the amino acid residues at sequence positions 187, 238 and 240 corresponding to the sequence position of SEQ ID NO.: 1 are mutated, preferably replaced by alanine (SEQ ID NO: 7) compared to the α-subunit of human Interleukin 27.

In a most preferred aspect, the present invention provides a mutein of the α-subunit of human Interleukin 27 comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1 of the α-subunit of human Interleukin 27, wherein in this mutein the amino acid residues at sequence positions 238 and 240 corresponding to the sequence position of SEQ ID NO.: 1 are mutated, preferably replaced by alanine (SEQ ID NO: 8) compared to the α-subunit of human Interleukin 27.

By “identity” or “sequence identity” is meant a property of sequences that measures their similarity or relationship. The term “sequence identity” or “identity” as used in the present invention means the percentage of pair-wise identical residues—following (homology) alignment of a sequence of a polypeptide of the invention with a sequence in question—with respect to the number of residues in the longer of these two sequences. Identity is measured by dividing the number of identical residues by the total number of residues and multiplying the product by 100.

The term “homology” is used herein in its usual meaning and includes identical amino acids as well as amino acids which are regarded to be conservative substitutions (for example, exchange of a glutamate residue by an aspartate residue) at equivalent positions in the linear amino acid sequence of a polypeptide of the disclosure (e.g., any lipocalin mutein of the disclosure).

The percentage of sequence homology or sequence identity can, for example, be determined herein using the program BLASTP, version blastp 2.2.5 (Nov. 16, 2002; cf Altschul, S. F. et al. (1997) Nucl. Acids Res. 25, 3389-3402). In this embodiment the percentage of homology is based on the alignment of the entire polypeptide sequences (matrix: BLOSUM 62; gap costs: 11.1; cutoff value set to 10-3) including the respective sequences. It is calculated as the percentage of numbers of “positives” (homologous amino acids) indicated as result in the BLASTP program output divided by the total number of amino acids selected by the program for the alignment.

The present invention also provides a mutein of human IL-27, comprising an α-subunit p28 and a β-subunit EBI3, wherein the α-subunit is a mutein of the α-subunit of human IL-27 as described herein. In a further embodiment thereof, the α-subunit is a mutein comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 1 of the α-subunit of human Interleukin 27 as described herein. The β-subunit EBI3 of the mutein of human IL-27 as disclosed herein may also comprise an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO.: 12 of the β-subunit of human Interleukin 27, preferably it refers to SEQ ID NO.: 12. In such a mutein of human IL-27 of the present invention at least one of the amino acid residues at sequence positions 187, 238 and 240 of the α-subunit corresponding to the sequence position of SEQ ID NO.: 1 can be mutated. In line with the above, in the mutein of human IL-27 of the present invention, the mutein of the corresponding α-subunit can comprise a single mutation at one of these sequence positions, but also a mutation at two or all three of these sequence positions. Thus, the disclosure with regard to the mutation of the α-subunit of human IL-27 at at least one of the amino acid residues at sequence positions 187, 238 and 240 corresponding to the sequence position of SEQ ID NO.: 1 is also applicable to the mutein of human IL-27.

Thus, according to the present invention in the mutein of human IL-27 of the present invention the amino acid residue at sequence position 187 of the α-subunit of human IL-27 corresponding to the sequence position of SEQ ID NO.: 1 can be replaced by alanine. In addition or alternatively, in the mutein of human IL-27 of the present invention the amino acid residue at sequence position 238 of the α-subunit of human IL-27 corresponding to the sequence position of SEQ ID NO.: 1 can be replaced by alanine. In addition or alternatively, in the mutein of human IL-27 of the present invention the amino acid residue at sequence position 240 of the α-subunit of human IL-27 corresponding to the sequence position of SEQ ID NO.: 1 can be replaced by alanine.

It is also comprised according to the present invention that in the mutein of human IL-27 of the present invention the amino acid residues at sequence positions 187 and 238 of the α-subunit of human IL-27 corresponding to the sequence position of SEQ ID NO.: 1 can be replaced by alanine. In yet another embodiment, in the mutein of human IL-27 of the present invention the amino acid residues at sequence positions 187 and 240 of the α-subunit of human IL-27 corresponding to the sequence position of SEQ ID NO.: 1 can also be replaced by alanine.

Most preferably, in the mutein of human IL-27 of the present invention the amino acid residues at sequence positions 238 and 240 of the α-subunit of human IL-27 corresponding to the sequence position of SEQ ID NO.: 1 are replaced by alanine.

It is within the scope of the present invention, that the above mentioned mutations of the human IL-27 at the amino acid residues 187, 238 and 240 corresponding to the sequence position of SEQ ID NO.: 1 to alanine can form muteins with 1, 2, or 3 alanines at any of the mentioned positions 187, 238 and 240 of human IL-27.

In yet another embodiment, in the mutein of human Interleukin 27 of the present invention the amino acid residue at at least one of sequence positions 187, 238 and 240 corresponding to the sequence position of SEQ ID NO.: 1 can be replaced by any amino acid, which cannot be O-glycosylated, thus any amino acid except serine or threonine.

The mutein of human IL-27 of the present invention may also further comprise one or more disulfide-bridges as explained above. Additionally or alternatively, the mutein of human IL-27 of the present invention can further comprise one or more salt bridges as explained above.

The present invention also provides a nucleic acid molecule comprising a nucleotide sequence encoding the mutein of human IL-27 of the present invention or the mutein of the α-subunit of human IL-27 of the present invention. In a further embodiment thereof, the nucleic acid molecule comprises a nucleotide sequence encoding a mutein of the α-subunit of human Interleukin 27, wherein the mutein comprises at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the α-subunit of human Interleukin 27 as described herein.

A nucleic acid molecule according to the present invention may comprise a nucleotide sequence encoding a mutein of SEQ ID NO: 2, SEQ ID NO: 3, SEQ ID NO: 4, SEQ ID NO: 5, SEQ ID NO: 6, SEQ ID NO: 7, SEQ ID NO: 8 or SEQ ID NO: 11, preferably SEQ ID NO: 3, SEQ ID NO: 4, SEQ ID NO: 8, SEQ ID NO: 11.

It is preferred, that the nucleic acid molecule of the present invention is operably linked to a regulatory sequence to allow expression of the nucleic acid molecule. This regulatory sequence may comprise a promoter sequence. The term “promoter” or “promoter sequence” means a DNA sequence which initiates and directs the transcription of a gene into an RNA transcript in cells.

The nucleic acid molecule according to the present invention may be comprised in a vector. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome.

In yet another aspect the invention provides the nucleic acid molecule as described herein for use as a therapeutic agent.

The present invention also provides a host cell containing a nucleic acid molecule of the present invention as described above. A host cell can be any prokaryotic (e.g., E. coli) or eukaryotic cell (e.g., insect cells, yeast or mammalian cells). Preferably, the host cell is a eukaryotic cell.

The present invention also provides an immune modulator comprising a mutein of the present invention. An immune modulator is any protein, substance or composition that is able to carry out immunomodulation, which is the adjustment of the immune response to a desired level, as e.g. in immunopotentiation, immunosuppression, or induction of immunologic tolerance.

The present invention also provides the use of a mutein of the present invention (a mutein of the α-subunit of human IL-27 or a mutein of human IL-27 comprising the α-subunit) for the manufacture of a medicament for treating a disease in a mammal, preferably a human. Suitable diseases include, but are not limited to an infectious disease, an autoimmune disease, cancer, multiple sclerosis, a transplantation-related disease, such as Graft-versus-Host-disease, a chronic inflammatory disease, such as chronic inflammatory bowel disease, acute inflammatory disease, sepsis, septic shock, diabetes or asthma. Preferably, sepsis is seen as a suitable disease that is treated with said mutein of the present invention in a mammal (preferably a human).

The present invention also provides a mutein of the present invention (a mutein of the α-subunit of human IL-27 or a mutein of human IL-27 comprising the α-subunit) for use in the treatment of diseases including the afore-mentioned infectious disease, an autoimmune disease, cancer, multiple sclerosis, a transplantation-related disease, such as Graft-versus-Host-disease, a chronic inflammatory disease, such as chronic inflammatory bowel disease, acute inflammatory disease, sepsis, septic shock, diabetes or asthma. Again, sepsis is seen as a suitable disease that is treated with said mutein of the present invention in a mammal (preferably a human).

The present invention also provides a method of treating an IL-27-mediated disease (also referred to a IL-27-connected disease), preferably an infectious disease, an autoimmune disease, cancer, multiple sclerosis, a chronic inflammatory disease, such as chronic inflammatory bowel disease, acute inflammatory disease, sepsis, septic shock, diabetes or asthma in a mammal, comprising the step of administering a composition comprising a mutein of the α-subunit of human IL-27 of the present invention or a mutein of human IL-27 of the present invention to a mammal in need thereof. Preferably, the mammal is a human. In an even more preferred embodiment, sepsis is seen as a suitable disease that is treated with said mutein of the present invention in a mammal (preferably a human).

The present invention also provides a method of producing a mutein according to the present invention, comprising the steps of:

(a) introducing into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence mutating at least one amino acid residues of human IL-27 or of the α-subunit of human IL-27 or of the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide selected from the group consisting of sequence positions 187, 238 and 240, and
(b) introducing the obtained nucleic acid molecule for expression into a suitable host cell or into a suitable cell extract or cell lysate.

It is comprised by the present invention that in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residue at sequence position 187.

Additionally or alternatively, it is comprised by the present invention that in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residue at sequence position 238.

Additionally or alternatively, is comprised by the present invention that in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residue at sequence position 240.

In yet another embodiment of the present invention, in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residues at sequence positions 187 and 238.

In yet another embodiment of the present invention, in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residues at sequence positions 187 and 240.

In a preferred embodiment of the present invention, in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residues at sequence positions 238 and 240.

In an even more preferred embodiment of the present invention, in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residue at sequence position 187 to alanine.

Additionally or alternatively, in yet another even more preferred embodiment of the present invention, in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residue at sequence position 238 to alanine.

Additionally or alternatively, in yet another even more preferred embodiment of the present invention, in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residue at sequence position 240 to alanine.

In another embodiment of the present invention, in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residues at sequence positions 187 and 238 to alanine.

In another embodiment of the present invention, in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residues at sequence positions 187 and 240 to alanine.

In a most preferred embodiment of the present invention, in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residues at sequence positions 238 and 240 to alanine.

The present invention also comprises that in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating 1, 2, or even all 3 of the amino acid residues at sequence positions 187, 238 and 240 to alanine.

In yet another embodiment, in the method of producing a mutein according to the present invention, in step (a) into a nucleic acid molecule encoding the human IL-27 polypeptide or the human IL-27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100% sequence identity to the amino acid sequence of SEQ ID NO: 1 of the human Interleukin 27 α-subunit polypeptide a nucleotide sequence is introduced mutating the amino acid residues at at least one of sequence positions 187, 238 and 240 to any amino acid, that cannot be O-glycosylated, thus any amino acid except serine or threonine.

The present invention also provides a mutein of the α-subunit of human Interleukin 27 as defined elsewhere herein (which may also be called “deletion mutant”) having at least one of the amino acid residues of the α-subunit of human Interleukin 27 selected from the group consisting of sequence positions 238 and 240 corresponding to the sequence position of SEQ ID NO: 1 mutated, further comprising 1, 2, 3, 4, 5, 6, 7, 8, 9, 10, 11, 12, or 13 mutations at one or more positions of any one of position 229, 230, 231, 232, 233, 234, 235, 236, 237, 239, 241, 242, or 243 corresponding to the sequence position of SEQ ID NO: 1. Thus, the present invention may also provide a mutein of the α-subunit of human Interleukin 27 having the amino acid residue of the α-subunit of human Interleukin 27 at sequence position 238 corresponding to the sequence position of SEQ ID NO: 1 mutated, further comprising 1, 2, 3, 4, 5, 6, 7, 8, 9, 10, 11, 12, or 13 mutations at one or more positions of any one of position 229, 230, 231, 232, 233, 234, 235, 236, 237, 239, 241, 242, or 243 corresponding to the sequence position of SEQ ID NO: 1. Thus, the present invention may also provide a mutein of the α-subunit of human Interleukin 27 having the amino acid residue of the α-subunit of human Interleukin 27 at sequence position 240 corresponding to the sequence position of SEQ ID NO: 1 mutated, further comprising 1, 2, 3, 4, 5, 6, 7, 8, 9, 10, 11, 12, or 13 mutations at one or more positions of any one of position 229, 230, 231, 232, 233, 234, 235, 236, 237, 239, 241, 242, or 243 corresponding to the sequence position of SEQ ID NO: 1. Thus, the present invention may also provide a mutein of the α-subunit of human Interleukin 27 having the amino acid residues of the α-subunit of human Interleukin 27 at sequence positions 238 and 240 corresponding to the sequence position of SEQ ID NO: 1 mutated, further comprising 1, 2, 3, 4, 5, 6, 7, 8, 9, 10, 11, 12, or 13 mutations at one or more positions of any one of position 229, 230, 231, 232, 233, 234, 235, 236, 237, 239, 241, 242, or 243 corresponding to the sequence position of SEQ ID NO: 1. Said mutant as defined in the following is again derived from the WT human Interleukin 27 (IL-27) α-subunit having SEQ ID NO: 1 as it is defined by the present invention. Thus, the present invention provides a mutein of the α-subunit of human Interleukin 27 as defined elsewhere herein, further comprising a mutated amino acid residue at one or more positions corresponding to positions 229, 230, 231, 232, 233, 234, 235, 236, 237, 239, 241, 242, or 243 of SEQ ID NO: 1. Each combination of mutated amino acids which refers to the additional mutations at one or more positions of any one of 229, 230, 231, 232, 233, 234, 235, 236, 237, 239, 241, 242, or 243 corresponding to the sequence position of SEQ ID NO: 1 may be combined herein with the disclosure of the mutein of the present invention defined as the mutein having at least one of the amino acid residues of the α-subunit of human Interleukin 27 selected from the group consisting of sequence positions 238 and 240 corresponding to the sequence position of SEQ ID NO: 1 mutated such as having the amino acid residue of the α-subunit of human Interleukin 27 at sequence position 238 corresponding to the sequence position of SEQ ID NO: 1 mutated or having the amino acid residue of the α-subunit of human Interleukin 27 at sequence position 240 corresponding to the sequence position of SEQ ID NO: 1 mutated or even having the amino acid residues of the α-subunit of human Interleukin 27 at sequence positions 238 and 240 corresponding to the sequence position of SEQ ID NO: 1 mutated.

In a further aspect, the present invention provides a mutein of the α-subunit of human Interleukin 27 as defined elsewhere herein comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100%, preferably at least 76% sequence identity to the amino acid sequence of SEQ ID NO: 1 of the α-subunit of human Interleukin 27, further comprising 1, 2, 3, 4, 5, 6, 7, 8, 9, 10, 11, 12, or 13 mutations at one or more positions of any one of 229, 230, 231, 232, 233, 234, 235, 236, 237, 239, 241, 242, or 243 corresponding to the sequence position of SEQ ID NO: 1.

Thus, the mutein as defined herein having at least one of the amino acid residues of the α-subunit of human Interleukin 27 selected from the group consisting of sequence positions 238 and 240 corresponding to the sequence position of SEQ ID NO: 1 mutated such as having the amino acid residue of the α-subunit of human Interleukin 27 at sequence position 238 or 240 corresponding to the sequence position of SEQ ID NO: 1 mutated or having the amino acid residues of the α-subunit of human Interleukin 27 at sequence positions 238 and 240 corresponding to the sequence position of SEQ ID NO: 1 mutated, further comprising a mutation (a mutated amino acid residue) at position 229 corresponding to the sequence position of SEQ ID NO: 1 may also be envisaged herein. The present invention may also comprise the mutein as defined herein further comprising a mutation at position 230 corresponding to the sequence position of SEQ ID NO: 1. The present invention may also comprise the mutein as defined herein further comprising a mutation at position 231 corresponding to the sequence position of SEQ ID NO: 1. The present invention may also comprise the mutein as defined herein further comprising a mutation at position 232 corresponding to the sequence position of SEQ ID NO: 1. The present invention may also comprise the mutein as defined herein further comprising a mutation at position 233 corresponding to the sequence position of SEQ ID NO: 1. The present invention may also comprise the mutein as defined herein further comprising a mutation at position 234 corresponding to the sequence position of SEQ ID NO: 1. The present invention may also comprise the mutein as defined herein further comprising a mutation at position 235 corresponding to the sequence position of SEQ ID NO: 1. The present invention may also comprise the mutein as defined herein further comprising a mutation at position 236 corresponding to the sequence position of SEQ ID NO: 1. The present invention may also comprise the mutein as defined herein further comprising a mutation at position 237 corresponding to the sequence position of SEQ ID NO: 1. The present invention may also comprise the mutein as defined herein further comprising a mutation at position 239 corresponding to the sequence position of SEQ ID NO: 1. The present invention may also comprise the mutein as defined herein further comprising a mutation at position 241 corresponding to the sequence position of SEQ ID NO: 1. The present invention may also comprise the mutein as defined herein further comprising a mutation at position 242 corresponding to the sequence position of SEQ ID NO: 1. The present invention may also comprise the mutein as defined herein further comprising a mutation at position 243 corresponding to the sequence position of SEQ ID NO: 1. Each embodiment of each additional mutation as defined herein may be combined with the disclosure regarding an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100%, preferably at least 76% sequence identity to the amino acid sequence of SEQ ID NO: 1 of the α-subunit of human Interleukin 27.

The present invention may also provide a mutein of the α-subunit of human Interleukin 27 having at least one of the amino acid residues of the α-subunit of human Interleukin 27 selected from the group consisting of sequence positions 238 and 240 corresponding to the sequence position of SEQ ID NO: 1 mutated, wherein at least the residues at amino acid positions 234 to 238 corresponding to the sequence position of SEQ ID NO: 1 are mutated. The present invention may also provide a mutein of the α-subunit of human Interleukin 27 having the amino acid residue of the α-subunit of human Interleukin 27 at sequence position 238 corresponding to the sequence position of SEQ ID NO: 1 mutated and wherein at least the residues at amino acid positions 234 to 238 corresponding to the sequence position of SEQ ID NO: 1 are mutated. The present invention may also provide a mutein of the α-subunit of human Interleukin 27 having the amino acid residue of the α-subunit of human Interleukin 27 at sequence position 240 corresponding to the sequence position of SEQ ID NO: 1 mutated and wherein additionally at least the residues at amino acid positions 234 to 238 corresponding to the sequence position of SEQ ID NO: 1 are mutated. The present invention may also provide a mutein of the α-subunit of human Interleukin 27 having the amino acid residues of the α-subunit of human Interleukin 27 at sequence positions 238 and 240 corresponding to the sequence position of SEQ ID NO: 1 mutated and wherein additionally at least the residues at amino acid positions 234 to 238 corresponding to the sequence position of SEQ ID NO: 1 are mutated. This means that the mutein as defined elsewhere herein further comprises 4 mutations at positions 234, 235, 236, 237, and 238 corresponding to the sequence position of SEQ ID NO: 1.

In a further aspect, the present invention provides a mutein of the α-subunit of human Interleukin 27 as defined herein comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100%, preferably at least 76% sequence identity to the amino acid sequence of SEQ ID NO: 1 of the α-subunit of human Interleukin 27, wherein at least the residues at amino acid positions 234 to 238 corresponding to the sequence position of SEQ ID NO: 1 are mutated compared to the α-subunit of human Interleukin 27. Said mutations at at least the residues at amino acid positions 234 to 238 of SEQ ID NO: 1 remove glycosylation sites (e.g. at position 238 of SEQ ID NO: 1 for example) but may also at the same time remove flexible, possibly destabilizing areas. This does not exclude any additional mutation(s) at other amino acid residues corresponding to the sequence position of SEQ ID NO: 1 except for the ones mentioned herein as long as the functionality of said protein or where applicable secretion-competence of said protein is not affected. When the mutein of the α-subunit of human Interleukin 27 as described herein refers to the polypeptide sequence of SEQ ID NO: 1 comprising the substitution of leucine 162 with cysteine (L162C) and comprising additional mutations (in this case deletions) at one or more positions of any one of 229, 230, 231, 232, 233, 234, 235, 236, 237, 239, 241, 242, or 243 corresponding to the sequence position of SEQ ID NO: 1 and/or at at least the residues at amino acid positions 234 to 238 corresponding to the sequence position of SEQ ID NO: 1 (such as at positions 234, 235, 236, 237 and 238), said mutein is secretion-competent. When the mutein of the α-subunit of human Interleukin 27 as described herein refers to the polypeptide sequence comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100%, preferably at least 76% sequence identity to the amino acid sequence of SEQ ID NO: 1 of the α-subunit of human Interleukin 27, wherein the residue at amino acid position 162 (leucine) is replaced by cysteine (L162C), compared to the α-subunit of human Interleukin 27 and additionally comprises mutations (in this case deletions) at one or more positions of any one of 229, 230, 231, 232, 233, 234, 235, 236, 237, 239, 241, 242, or 243 corresponding to the sequence position of SEQ ID NO: 1 and/or at at least the residues at amino acid positions 234 to 238 corresponding to the sequence position of SEQ ID NO: 1 (such as at positions 234, 235, 236, 237 and 238), said mutein may also be secretion-competent as defined elsewhere herein. These particular mutations (e.g. deletions) result in the prevention of O-glycosylation as described elsewhere herein which is important for the improved homogeneity while maintaining complete functionality and in some cases even enhanced activity of the protein, more compact folding or less accessibility for proteolysis. In this context, not only a mutation (e.g. deletion) of the residues at amino acid positions 234 to 238 corresponding to the sequence position of SEQ ID NO: 1, such as a mutation (e.g. deletion) of the residues at amino acid positions 234, 235, 236, 237 and 238 corresponding to the sequence position of SEQ ID NO: 1, is possible, which comprises Thr238 comprising a O-glycosylation site, but also a mutation (in this case deletion) of the residues at amino acid positions 234 to 239 corresponding to the sequence position of SEQ ID NO: 1 (such as at positions 234, 235, 236, 237, 238 and 239), 234 to 240 corresponding to the sequence position of SEQ ID NO: 1 (such as at positions 234, 235, 236, 237, 238, 239 and 240), 234 to 241 corresponding to the sequence position of SEQ ID NO: 1 (such as at positions 234, 235, 236, 237, 238, 239, 240 and 241), 234 to 242 corresponding to the sequence position of SEQ ID NO: 1 (such as at positions 234, 235, 236, 237, 238, 239, 240, 241 and 242) or 234 to 243 corresponding to the sequence position of SEQ ID NO: 1 (such as at positions 234, 235, 236, 237, 238, 239, 240, 241, 242 and 243). Thus, in this context the term “at least” with regard to “at least the residues at amino acid positions 234 to 238 corresponding to the sequence position of SEQ ID NO: 1” also comprises the other deletion variations as mentioned.

The present invention also comprises a mutein of the α-subunit of human Interleukin 27 as defined herein having at least one of the amino acid residues of the α-subunit of human Interleukin 27 selected from the group consisting of sequence positions 238 and 240 corresponding to the sequence position of SEQ ID NO: 1 mutated such as having the amino acid residue of the α-subunit of human Interleukin 27 at sequence position 238 or 240 corresponding to the sequence position of SEQ ID NO: 1 mutated or having the amino acid residues of the α-subunit of human Interleukin 27 at sequence positions 238 and 240 corresponding to the sequence position of SEQ ID NO: 1 mutated, and wherein at least the residues at amino acid positions 234 to 239 corresponding to the sequence position of SEQ ID NO: 1, at least the residues at amino acid positions 234 to 240 corresponding to the sequence position of SEQ ID NO: 1, at least the residues at amino acid positions 234 to 241 corresponding to the sequence position of SEQ ID NO: 1, at least the residues at amino acid positions 234 to 242 corresponding to the sequence position of SEQ ID NO: 1, or even the residues at amino acid positions 234 to 243 corresponding to the sequence position of SEQ ID NO: 1 are mutated. Thus, the present invention also provides a mutein of the α-subunit of human Interleukin 27 as defined above comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100%, preferably at least 76% sequence identity to the amino acid sequence of SEQ ID NO: 1 of α-subunit of human Interleukin 27, wherein at least the residues at amino acid positions 234 to 239 corresponding to the sequence position of SEQ ID NO: 1 (such as at positions 234, 235, 236, 237, 238 and 239), at amino acid positions 234 to 240 corresponding to the sequence position of SEQ ID NO: 1 (such as at positions 234, 235, 236, 237, 238, 239 and 240), at amino acid positions 234 to 241 corresponding to the sequence position of SEQ ID NO: 1 (such as at positions 234, 235, 236, 237, 238, 239, 240 and 241), at amino acid positions 234 to 242 corresponding to the sequence position of SEQ ID NO: 1 (such as at positions 234, 235, 236, 237, 238, 239, 240, 241 and 242), at amino acid positions 234 to 243 corresponding to the sequence position of SEQ ID NO: 1 (such as at positions 234, 235, 236, 237, 238, 239, 240, 241, 242 and 243) are mutated.

The present invention also provides a mutein of the α-subunit of human Interleukin 27 as defined herein having at least one of the amino acid residues of the α-subunit of human Interleukin 27 selected from the group consisting of sequence positions 238 and 240 corresponding to the sequence position of SEQ ID NO: 1 mutated, and wherein the residues at amino acid positions 229 to 243 corresponding to the sequence position of SEQ ID NO: 1 (such as at positions 229, 230, 231, 232, 233, 234, 235, 236, 237, 238, 239, 240, 241, 242, 243) are mutated (see FIG. 13). A deletion of the residues at amino acid positions 229 to 243 corresponding to the sequence position of SEQ ID NO: 1 may refer to C-terminal region of said IL-27α. Again, said mutations at the residues at amino acid positions 229 to 243 corresponding to the sequence position of SEQ ID NO: 1 remove glycosylation sites (e.g. at position 238 and 240 corresponding to the sequence position of SEQ ID NO: 1 for example) but may also at the same time remove flexible, possibly destabilizing areas. In another aspect, the present invention also provides a mutein of the α-subunit of human Interleukin 27 as defined herein comprising an amino acid sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100%, preferably at least 76% sequence identity to the amino acid sequence of SEQ ID NO: 1 of the α-subunit of human Interleukin 27, wherein the residues at amino acid positions 229 to 243 corresponding to the sequence position of SEQ ID NO: 1 are mutated (SEQ ID NO: 11) as defined herein (see FIG. 13).

Each disclosure made for the human Interleukin 27 (IL-27) alpha-subunit or for the human Interleukin 27 (IL-27) comprising said alpha-subunit as defined elsewhere herein (also including the disclosure to the nucleic acid molecule, the host cell, the immune modulator, the first and second medical uses, and the method of producing such mutein) may also be applicable for the deletion mutant.

In this context, the method of producing said mutein as defined herein also provides, wherein in step (a) into a nucleic acid molecule encoding the human Interleukin 27 polypeptide or the human Interleukin 27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100%, preferably at least 76% sequence identity to the amino acid sequence of SEQ ID NO: 1 of the human Interleukin 27 α-subunit polypeptide a nucleotide sequence is introduced further mutating 1, 2, 3, 4, 5, 6, 7, 8, 9, 10, 11, 12 or 13 times at one or more positions of any one of 229, 230, 231, 232, 233, 234, 235, 236, 237, 239, 241, 242, or 243 corresponding to the sequence position of SEQ ID NO: 1.

Also provided herein is the method of producing said mutein as defined herein, wherein in step (a) into a nucleic acid molecule encoding the human Interleukin 27 polypeptide or the human Interleukin 27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100%, preferably at least 76% sequence identity to the amino acid sequence of SEQ ID NO: 1 of the human Interleukin 27 α-subunit polypeptide a nucleotide sequence is introduced mutating at least the residues at amino acid positions 234 to 238 corresponding to the sequence position of SEQ ID NO: 1, at least the residues at amino acid positions 234 to 239 corresponding to the sequence position of SEQ ID NO: 1, at least the residues at amino acid positions 234 to 240 corresponding to the sequence position of SEQ ID NO: 1, at least the residues at amino acid positions 234 to 241 corresponding to the sequence position of SEQ ID NO: 1, at least the residues at amino acid positions 234 to 242 corresponding to the sequence position of SEQ ID NO: 1, or at least the residues at amino acid positions 234 to 243 corresponding to the sequence position of SEQ ID NO: 1.

Also provided herein is the method of producing said mutein as defined herein, wherein in step (a) into a nucleic acid molecule encoding the human Interleukin 27 polypeptide or the human Interleukin 27 α-subunit polypeptide or the polypeptide comprising at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or even 100%, preferably at least 76% sequence identity to the amino acid sequence of SEQ ID NO: 1 of the human Interleukin 27 α-subunit polypeptide a nucleotide sequence is introduced mutating the residues at amino acid positions 229 to 243 corresponding to the sequence position of SEQ ID NO: 1 (SEQ ID NO: 11).

The invention is further illustrated by the following experimental Examples.

### EXAMPLES

Sequence and Structural Alignments:

DNA sequence alignments were performed with Clustal Omega¹⁸. The i-Tasser program¹⁹ was used for homology modelling of the human IL-27 alpha^{L162C} structure. Structure alignments and analyses were performed with Yasara Structure (www.yasara.org).

Generation of IL-27 Mutants:

Genes for human and murine IL-27 alpha were amplified by PCR from their cDNA (Origene) and cloned into the pSVL vector (Amersham) after restriction digestion. Mutants were generated by site-directed mutagenesis.

Cell Culture Experiments:

293T cells were cultured in Dulbecco's modified Eagle's Medium (DMEM), which contained L-Ala-L-Gln (AQmedia, Sigma-Aldrich) and 10% (v/v) fetal bovine serum (biochrome), at 37° C. and 5% CO2. 293T cell medium was mixed with a 1% (v/v) antibiotic antifungal solution (25 μg/ml amphotericin B, 10 mg/ml streptomycin and 10,000 units penicillin; Sigma-Aldrich). Transient transfections were performed for 24 h in p35 poly-D-lysine coated shells (Becton Dickinson) with GeneCellin (BioCellChallenge) according to the manufacturer's protocol. Equal amounts of constructs (alpha, beta or empty vector) were transfected with a total amount of 2 μg DNA (p35 dishes). For secretion experiments, the cells were transfected for 8 h, then washed twice with PBS and incubated with 0.5 ml fresh medium for another 16 h. Before lysis, cells were washed twice with ice-cold PBS. Cell lysis was performed with RIPA buffer (50 mM Tris/HCl, pH 7.5, 150 mM NaCl, 1.0% Nonidet P40 substitute, 0.5% sodium deoxycholate, 0.1% SDS, lx Roche complete protease inhibitor w/o EDTA; Roche Diagnostics). The medium was centrifuged for 5 min at 300 g and 4° C. for the analysis of secreted proteins. Subsequently, the samples were mixed with 0.1 volume 500 mM Tris/HCl, pH 7.5, 1.5 M NaCl and protease inhibitor and centrifuged for 15 min at 20,000 g and 4° C. The samples were then analyzed for the presence of the protein. 0.2 volumes of 5× Laemmli, which contained β-mercaptoethanol for reducing SDS-PAGE, were added to the samples. Deglycosylation experiments were performed according to the manufacturer's instructions (NEB).

Recombinant Protein Production:

The human IL-27α cDNA optimized for expression in E. coli (without ER import sequence) was acquired from GeneArt and cloned into the pET21a vector (Merck Millipore) with an N-terminal hexa-histidine tag and a TEV protease cleavage site after the tag. The L156C mutation (corresponding to L162C mutation in the human sequence) was inserted by site-directed mutagenesis. The reference protein was expressed as inclusion bodies in selective LB medium. The culture was induced at OD600=0.6 with 1 mM IPTG and harvested after another 4 h by centrifugation (5,000 rpm, 15 min, 4° C.). To isolate the inclusion bodies, the cells were lysed on ice using ultrasound in 100 mM Tris/HCl, pH 7.5, 100 mM NaCl, 5 mM EDTA, SigmaFAST protease inhibitor and then centrifuged (20,000 g, 20 min, 4° C.). The pellet was resuspended and washed twice with 100 mM Tris/HCl, pH 7.5, 500 mM NaCl, 5 mM EDTA, 1.0% Triton X-100 and again with 100 mM Tris/HCl, pH 7.5, 100 mM NaCl. The inclusion bodies were then solubilized in 50 mM sodium phosphate, pH 7.5, 250 mM NaCl, 6 M GdmCl and 10 mM β-mercaptoethanol at 4° C. After overnight solubilization, the solution was centrifuged (20,000 g, 20 min, 20° C.). The supernatant was diluted with one volume unit of 50 mM sodium phosphate, pH 7.5, 250 mM NaCl, 5 M GdmCl and loaded onto a Ni-Sepharose HP column (GE Healthcare). Bound protein was washed with 50 mM sodium phosphate, pH 7.5, 250 mM NaCl, 5 M GdmCl, 30 mM imidazole, 1 mM DTT and eluted with 50 mM sodium phosphate, pH 3.5, 250 mM NaCl, 5 M GdmCl and 1 mM DTT. Eluted protein was further purified by gel filtration (HiPrep 16/60 Sephacryl S-400 HR column (GE Healthcare)) and buffer changed into 50 mM IVIES pH 6.0, 6 M Urea, 1 mM EDTA. The protein concentration was determined spectrophotometrically at A280 nm. Human IL-27α^{L162C} cDNA optimized for expression in H. sapiens was ordered from GeneArt (Thermo Fisher Scientific) in a pcDNA3.4 TOPO vector for mammalian cell expression. The T238A and S240A mutations were inserted by site-directed mutagenesis. Protein expression was performed with the Expi293 expression system according to the manufacturer's specifications (Thermo Fisher Scientific). 48 h post-transfection, the medium was harvested by centrifugation (300 g, 15 min, 4° C.), concentrated to 4.1 μg/ml by Vivaspin 20 10 kDa centrifugal units (VWR) and used for immunological assays. hIL-27αL156CHis6 purified from E. coli inclusion bodies was used as a reference to establish a linear fit standard curve for the quantification of hIL-27α^{L162C,T238A,S240A} in Expi293 supernatants using immunoblot signals.

Quantification:

Western blots were quantified with the Bio-1D software (Vilber Lourmat). For the quantification of P-STAT immunoblots the Western blot signals of cells treated with IL-27alpha^{L162C,T238A,S240A} were normalized to the IL-27alpha^{L162C} or IL-27alpha signal. All experiments were performed at least twice and a representative experiment was selected.

Activity Assays:

STAT experiments were performed with the human Burkitt lymphoma BL-2 cell line (DKSM). Before use, BL-2 cells were cultured overnight in serum-free RPMI-1640 medium. Cells were incubated in 48-well plates (2×106 cells/well) in RPMI-1640 medium with 0.5% BSA for 60 min with 1000 ng/mL hIL-27α^{L162C}, hIL-27α, hIL-27alpha^{L162C,T238A,S240A}, hIL-27alpha^{T238A,S240A (=ΔO)} and hEBI3, hEBI3^{N55QN105Q (=ΔN)} Expi293 supernatant or non-transfected control Expi293 supernatant in 48-well plates (2×106 cells/well). The reaction was stopped by diluting the cells with ice-cold PBS buffer and lysis in NP40 lysis buffer (with protease and phosphatase inhibitors). Phosphorylates and total STAT protein was detected by immunoblotting. Rabbit antibodies from Cell Signaling Technology were used (P-STAT1, #9167; STAT1, #9172) (see FIGS. 10 and 12).

C-Terminal Truncation in IL-27α L162C:

1×10⁴ STAT1 Luciferase Reporter HeLa cells (Signosis, SL-0004-NP) were seeded in 100 μl DMEM (Sigma Aldrich) containing 0.1% FCS per well on a 96-well plate. After incubation over night, 60 μl medium was replaced with 60 μl cleared supernatant from Expi293 cells, which have been either mock transfected (vehicle control) or transfected to transiently express and secrete hIL-27α^{L162C} (full length construct) or hIL-27α^{L162C,AC} (construct lacking the C-terminal region, H229-P243). After stimulation for 22 h, cells were washed with PBS and subjected to luciferase assays according to the manufacturer's protocol (Promega, E1500). Data was normalized to full length hIL-27α^{L162C} (n=3 biological replicates, each as mean of 4 technical replicates) (see FIG. 13).

The invention is further characterized by the following items:

### Items

1. A mutein of the α-subunit of human Interleukin 27 (SEQ ID NO: 1), wherein at least one of the amino acid residues of the α-subunit of human Interleukin 27 (SEQ ID NO: 1) selected from the group consisting of sequence positions 187, 238 and 240 is/are mutated.
2. The mutein of item 1, wherein the amino acid residue of the α-subunit of human Interleukin 27 (SEQ ID NO: 1) at sequence position 187 is replaced by alanine (SEQ ID NO: 2).
3. The mutein of item 1 or 2, wherein the amino acid residue of the α-subunit of human Interleukin 27 (SEQ ID NO: 1) at sequence position 238 is replaced by alanine (SEQ ID NO: 3).
4. The mutein of any of the preceding items, wherein the amino acid residue of the α-subunit of human Interleukin 27 (SEQ ID NO: 1) at sequence position 240 is replaced by alanine (SEQ ID NO: 4).
5. The mutein of any of the preceding items, wherein the amino acid residues of the α-subunit of human Interleukin 27 (SEQ ID NO: 1) at sequence positions 187 and 238 are replaced by alanine (SEQ ID NO: 5).
6. The mutein of any of the preceding items, wherein the amino acid residues of the α-subunit of human Interleukin 27 (SEQ ID NO: 1) at sequence positions 187 and 240 are replaced by alanine (SEQ ID NO: 6).
7. The mutein of any of the preceding items, wherein the amino acid residues of the α-subunit of human Interleukin 27 (SEQ ID NO: 1) at sequence positions 187, 238 and 240 are replaced by alanine (SEQ ID NO: 7).
8. The mutein of any of the preceding items, wherein the amino acid residues of the α-subunit of human Interleukin 27 (SEQ ID NO: 1) at sequence positions 238 and 240 are replaced by alanine (SEQ ID NO: 8).
9. The mutein of any of the preceding items, wherein the mutein further comprises one or more salt bridges.
10. The mutein of any of the preceding items, wherein the mutein further comprises one or more disulfide-bridges.
11. A mutein of human Interleukin 27, comprising an α-subunit p28 and a β-subunit Ebi3, wherein the α-subunit is a mutein of the α-subunit of human Interleukin 27 (SEQ ID NO: 1) of any of items 1 to 10.
12. The mutein of item 11, wherein at least one of the amino acid residues of the α-subunit of human Interleukin 27 (SEQ ID NO: 1) selected from the group consisting of sequence positions 187, 238 and 240 is/are mutated.
13. The mutein of any of items 11 to 12, wherein the amino acid residue of the α-subunit of human Interleukin 27 (SEQ ID NO: 1) at sequence position 187 is replaced by alanine.
14. The mutein of any of items 11 to 13, wherein the amino acid residue of the α-subunit of human Interleukin 27 (SEQ ID NO: 1) at sequence position 238 is replaced by alanine.
15. The mutein of any of items 11 to 14, wherein the amino acid residue of the α-subunit of human Interleukin 27 (SEQ ID NO: 1) at sequence position 240 is replaced by alanine.
16. The mutein of any of items 11 to 15, wherein the amino acid residues of the α-subunit of human Interleukin 27 (SEQ ID NO: 1) at sequence positions 187 and 238 are replaced by alanine.
17. The mutein of any of items 11 to 16, wherein the amino acid residues of the α-subunit of human Interleukin 27 (SEQ ID NO: 1) at sequence positions 187 and 240 are replaced by alanine.
18. The mutein of any of items 11 to 17, wherein the amino acid residues of the α-subunit of human Interleukin 27 (SEQ ID NO: 1) at sequence positions 187, 238 and 240 are replaced by alanine.
19. The mutein of any of items 11 to 18, wherein the amino acid residues of the α-subunit of human Interleukin 27 (SEQ ID NO: 1) at sequence positions 238 and 240 are replaced by alanine.
20. The mutein of any of items 11 to 19, wherein the mutein further comprises one or more salt bridges.
21. The mutein of any of items 11 to 20, wherein the mutein further comprises one or more disulfide-bridges.
22. A nucleic acid molecule comprising a nucleotide sequence encoding the mutein of human Interleukin 27 or the mutein of the α-subunit of human Interleukin 27 of any of items 1 to 21.
23. The nucleic acid molecule of item 22, comprising a nucleotide sequence encoding a mutein of SEQ ID NO: 2, SEQ ID NO: 3, SEQ ID NO: 4, SEQ ID NO: 5, SEQ ID NO: 6, SEQ ID NO: 7, or SEQ ID NO: 8.
24. The nucleic acid molecule of any of items 22 to 23, wherein the nucleic acid molecule is operably linked to a regulatory sequence to allow expression of the nucleic acid molecule.
25. The nucleic acid molecule of item 24, wherein the regulatory sequence comprises a promoter sequence.
26. The nucleic acid molecule of any of items 22 to 25 comprised in a vector.
27. A host cell containing a nucleic acid molecule of any of items 22 to 26.
28. An immune modulator comprising a mutein of any of items 1 to 21.
29. Use of a mutein of any of items 1 to 21 for the manufacture of a medicament for treating an infectious disease, an autoimmune disease, multiple sclerosis, cancer, a transplantation-related disease, such as Graft-versus-Host-disease, a chronic inflammatory disease, such as chronic inflammatory bowel disease, acute inflammatory disease, sepsis, septic shock, diabetes or asthma in a mammal.
30. The mutein of any of items 1 to 21 for use in therapy.
31. The mutein of any of items 1 to 21 for use in the treatment of an infectious disease, an autoimmune disease, multiple sclerosis, cancer, a transplantation-related disease, such as Graft-versus-Host-disease, a chronic inflammatory disease, such as chronic inflammatory bowel disease, acute inflammatory disease, sepsis, septic shock, diabetes or asthma in a mammal.
32. A method of treating an Interleukin 27-mediated disease, preferably an infectious disease, an autoimmune disease, multiple sclerosis, cancer, a transplantation-related disease, such as Graft-versus-Host-disease, a chronic inflammatory disease, such as chronic inflammatory bowel disease, acute inflammatory disease, sepsis, septic shock, diabetes or asthma in a mammal, comprising the step of administering a composition comprising a mutein of any of items 1 to 21 to a mammal in need thereof.
33. Method for producing a mutein of any of items 1 to 21, comprising the steps of:
a) introducing into a nucleic acid molecule encoding the human Interleukin 27 polypeptide or the human Interleukin 27 α-subunit polypeptide a nucleotide sequence mutating at least one amino acid residues of human Interleukin 27 or of the α-subunit of human Interleukin 27 selected from the group consisting of sequence positions 187, 238 and 240, and
b) introducing the obtained nucleic acid molecule of step (a) for expression into a suitable host cell or into a suitable cell extract or cell lysate.
34. The method of item 33, wherein in step (a) into a nucleic acid molecule encoding the human Interleukin 27 polypeptide or the human Interleukin 27 α-subunit polypeptide a nucleotide sequence is introduced mutating the amino acid residue of the α-subunit of human Interleukin 27 (SEQ ID NO: 1) at sequence position 187 to alanine.
35. The method of item 33 or 34, wherein in step (a) into a nucleic acid molecule encoding the human Interleukin 27 polypeptide or the human Interleukin 27 α-subunit polypeptide a nucleotide sequence is introduced mutating the amino acid residue of the α-subunit of human Interleukin 27 (SEQ ID NO: 1) at sequence position 238 to alanine.
36. The method of any of items 33 to 35, wherein in step (a) into a nucleic acid molecule encoding the human Interleukin 27 polypeptide or the human Interleukin 27 α-subunit polypeptide a nucleotide sequence is introduced mutating the amino acid residue of the α-subunit of human Interleukin 27 (SEQ ID NO: 1) at sequence position 240 to alanine.
37. The method of any of items 33 to 36, wherein in step (a) into a nucleic acid molecule encoding the human Interleukin 27 polypeptide or the human Interleukin 27 α-subunit polypeptide a nucleotide sequence is introduced mutating the amino acid residues of the α-subunit of human Interleukin 27 (SEQ ID NO: 1) at sequence positions 187 and 238 to alanine.
38. The method of any of items 33 to 37, wherein in step (a) into a nucleic acid molecule encoding the human Interleukin 27 polypeptide or the human Interleukin 27 α-subunit polypeptide a nucleotide sequence is introduced mutating the amino acid residues of the α-subunit of human Interleukin 27 (SEQ ID NO: 1) at sequence positions 187 and 240 to alanine.
39. The method of any of items 33 to 38, wherein in step (a) into a nucleic acid molecule encoding the human Interleukin 27 polypeptide or the human Interleukin 27 α-subunit polypeptide a nucleotide sequence is introduced mutating the amino acid residues of the α-subunit of human Interleukin 27 (SEQ ID NO: 1) at sequence positions 187, 238 and 240 to alanine.
40. The method of any of items 33 to 39, wherein in step (a) into a nucleic acid molecule encoding the human Interleukin 27 polypeptide or the human Interleukin 27 α-subunit polypeptide a nucleotide sequence is introduced mutating the amino acid residues of the α-subunit of human Interleukin 27 (SEQ ID NO: 1) at sequence positions 238 and 240 to alanine.
41. A mutein of the α-subunit of human Interleukin 27 (SEQ ID NO: 1), wherein at least the residues at amino acid positions 234 to 238 are deleted.

### REFERENCES

1. Vignali, D. A. & Kuchroo, V. K. IL-12 family cytokines: immunological playmakers. Nat Immunol 13, 722-8 (2012).
2. Langrish, C. L. et al. IL-12 and IL-23: master regulators of innate and adaptive immunity. Immunol Rev 202, 96-105 (2004).
3. Yoshida, H. & Hunter, C. A. The immunobiology of interleukin-27. Annu Rev Immunol 33, 417-43 (2015).
4. Pflanz, S. et al. IL-27, a heterodimeric cytokine composed of EBI3 and p28 protein, induces proliferation of naïve CD4(+) T cells. Immunity 16, 779-90 (2002).
5. Awasthi, A. et al. A dominant function for interleukin 27 in generating interleukin 10-producing anti-inflammatory T cells. Nat Immunol 8, 1380-9 (2007).
6. Fitzgerald, D. C. et al. Suppression of autoimmune inflammation of the central nervous system by interleukin 10 secreted by interleukin 27-stimulated T cells. Nat Immunol 8, 1372-9 (2007).
7. Stumhofer, J. S. et al. Interleukins 27 and 6 induce STAT3-mediated T cell production of interleukin 10. Nat Immunol 8, 1363-71 (2007).
8. Batten, M. et al. Interleukin 27 limits autoimmune encephalomyelitis by suppressing the development of interleukin 17-producing T cells. Nat Immunol 7, 929-36 (2006).
9. Stumhofer, J. S. et al. Interleukin 27 negatively regulates the development of interleukin 17-producing T helper cells during chronic inflammation of the central nervous system. Nat Immunol 7, 937-45 (2006).
10. Patel, D. D. & Kuchroo, V. K. Th17 Cell Pathway in Human Immunity: Lessons from Genetics and Therapeutic Interventions. Immunity 43, 1040-51 (2015).
11. Yoon, C. et al. Charged residues dominate a unique interlocking topography in the heterodimeric cytokine interleukin-12. EMBO J 19, 3530-41 (2000).
12. Lupardus, P. J. & Garcia, K. C. The structure of interleukin-23 reveals the molecular basis of p40 subunit sharing with interleukin-12. J Mol Biol 382, 931-41 (2008).
13. Wang, X. et al. A novel IL-23p19/Ebi3 (IL-39) cytokine mediates inflammation in Lupus-like mice. Eur J Immunol (2016).
14. Gubler, U. et al. Coexpression of two distinct genes is required to generate secreted bioactive cytotoxic lymphocyte maturation factor. Proc Natl Acad Sci USA 88, 4143-7 (1991).
15. Oppmann, B. et al. Novel p19 protein engages IL-12p40 to form a cytokine, IL-23, with biological activities similar as well as distinct from IL-12. Immunity 13, 715-25 (2000).
16. Reitberger, S., Haimerl, P., Aschenbrenner, I., Esser-von Bieren, J. & Feige, M. J. Assembly-induced folding regulates interleukin 12 biogenesis and secretion. J Biol Chem (2017).
17. Stumhofer, J. S. et al. A role for IL-27p28 as an antagonist of gp130-mediated signaling. Nat Immunol 11, 1119-26 (2010).
18. Sievers, F. et al. Fast, scalable generation of high-quality protein multiple sequence alignments using Clustal Omega. Mol Syst Biol 7, 539 (2011).
19. Zhang, Y. I-TASSER server for protein 3D structure prediction. BMC Bioinformatics 9, 40 (2008).
20. Yan, J., Mitra, A., Hu, J., Cutrera, J. J., Xia, X., Doetschman, T., Gagea, M., Mishra, L., and Li, S. (2016). Interleukin-30 (IL-27p28) alleviates experimental sepsis by modulating cytokine profile in NKT cells. J Hepatol 64, 1128-1136.
21. Müller, S., et al. (2019) A folding switch regulates interleukin 27 biogenesis and secretion of it's a subunit as a cytokine. PNAS.
22. Hebert et al. (2014), Nature chemical biology 10, 902-910
23. Mulagapati et al. (2017), Biochemistry 56, 1218-1226
24. Garbers et al. (2013), J Biol Chem 288(6): 4346-4354

1. A mutein of the α-subunit of human Interleukin 27, wherein at least one amino acid residues of the α-subunit of human Interleukin 27 at a position selected from the group consisting of sequence positions 238 and 240 is mutated.

2. The mutein of claim 1, wherein the amino acid residue of the α-subunit of human Interleukin 27 at sequence position 238 is mutated, the amino acid residue of the α-subunit of human Interleukin 27 at sequence position 240 is mutated, or the amino acid residues of the α-subunit of human Interleukin 27 at sequence positions 238 and 240 are each mutated.

3. (canceled)

4. (canceled)

5. The mutein of claim 1, wherein the amino acid residue of the α-subunit of human Interleukin 27 at sequence position 238 is replaced by alanine (SEQ ID NO: 3), the amino acid residue of the α-subunit of human Interleukin 27 at sequence position 240 is replaced by alanine (SEQ ID NO: 4), or the amino acid residues of the α-subunit of human Interleukin 27 at sequence positions 238 and 240 are each replaced by alanine (SEQ ID NO: 8).

6. (canceled)

7. (canceled)

8. The mutein of claim 1, further comprising 1, 2, 3, 4, 5, 6, 7, 8, 9, 10, 11, 12 or 13 mutations at one or more positions of any one of position 229, 230, 231, 232, 233, 234, 235, 236, 237, 239, 241, 242, or 243 and/or wherein at least the residues at amino acid positions 234 to 238, at least the residues at amino acid positions 234 to 239, at least the residues at amino acid positions 234 to 240, at least the residues at amino acid positions 234 to 241, at least the residues at amino acid positions 234 to 242, or at least the residues at amino acid positions 234 to 243 are mutated and/or wherein the residues at amino acid positions 229 to 243 are mutated (SEQ ID NO: 11) and/or wherein the mutein comprises at least 60% sequence identity to the α-subunit of human Interleukin 27.

9-11. (canceled)

12. The mutein of claim 1, wherein the mutein further comprises one or more salt bridges and/or wherein the mutein further comprises one or more disulfide-bridges.

13. (canceled)

14. A mutein of human Interleukin 27, comprising an α-subunit p28 and a β-subunit Ebi3, wherein the α-subunit is a mutein of the α-subunit of human Interleukin 27 of claim 1.

15. The mutein of claim 14, wherein the amino acid residue of the α-subunit of human Interleukin 27 at sequence position 238 is mutated, the amino acid residue of the α-subunit of human Interleukin 27 at sequence position 240 is mutated, or wherein the amino acid residues of the α-subunit of human Interleukin 27 at sequence positions 238 and 240 are each mutated.

16-18. (canceled)

19. The mutein of claim 14, wherein the amino acid residue of the α-subunit of human Interleukin 27 at sequence position 238 is replaced by alanine (SEQ ID NO: 3), the amino acid residue of the α-subunit of human Interleukin 27 at sequence position 240 is replaced by alanine (SEQ ID NO: 4), or the amino acid residues of the α-subunit of human Interleukin 27 at sequence positions 238 and 240 are each replaced by alanine (SEQ ID NO: 8).

20. (canceled)

21. (canceled)

22. The mutein of claim 14, further comprising 1, 2, 3, 4, 5, 6, 7, 8, 9, 10, 11, 12 or 13 mutations at one or more positions of any one of position 229, 230, 231, 232, 233, 234, 235, 236, 237, 239, 241, 242, or 243 and/or wherein at least the residues at amino acid positions 234 to 238, at least the residues at amino acid positions 234 to 239, at least the residues at amino acid positions 234 to 240, at least the residues at amino acid positions 234 to 241, at least the residues at amino acid positions 234 to 242, or at least the residues at amino acid positions 234 to 243 are mutated and/or wherein the residues at amino acid positions 229 to 243 are mutated and/or wherein the α-subunit comprises at least 60% sequence identity to the α-subunit of human Interleukin 27.

23-25. (canceled)

26. The mutein of claim 14, wherein the mutein further comprises one or more salt bridges and/or wherein the mutein further comprises one or more disulfide-bridges.

27. (canceled)

28. A nucleic acid molecule comprising a nucleotide sequence encoding the mutein of human Interleukin 27 or the mutein of the α-subunit of human Interleukin 27 of claim 1, optionally wherein the nucleotide sequence encodes the mutein of the α-subunit of human Interleukin 27, wherein said mutein comprises at least 60% sequence identity to the α-subunit of human Interleukin 27.

29. (canceled)

30. The nucleic acid molecule of claim 28, comprising a nucleotide sequence encoding a mutein of SEQ ID NO: 3, SEQ ID NO: 4, SEQ ID NO: 8 or SEQ ID NO: 11 and/or wherein the nucleic acid molecule is operably linked to a regulatory sequence to allow expression of the nucleic acid molecule, optionally wherein the regulatory sequence comprises a promoter sequence.

31. (canceled)

32. (canceled)

33. The nucleic acid molecule of claim 28 comprised in a vector.

34. A host cell containing a nucleic acid molecule of claim 28.

35. An immune modulator comprising a mutein of claim 1.

36. (canceled)

37. A method of treating an infectious disease, an autoimmune disease, multiple sclerosis, cancer, a transplantation-related disease, such as Graft-versus-Host-disease, a chronic inflammatory disease, such as chronic inflammatory bowel disease, acute inflammatory disease, sepsis, septic shock, diabetes or asthma, the method comprising administering an effective amount of the mutein of claim 1 to a mammal.

38. A method of producing a mutein of claim 1, comprising the steps of:
a) introducing into a nucleic acid molecule encoding the human Interleukin 27 polypeptide or the human Interleukin 27 α-subunit polypeptide or a polypeptide comprising at least 60% sequence identity to the human Interleukin 27 α-subunit polypeptide a nucleotide sequence mutating at least one amino acid residues of human Interleukin 27 or of the α-subunit of human Interleukin 27 or of a polypeptide comprising at least 60% sequence identity to the human Interleukin 27 α-subunit polypeptide selected from the group consisting of sequence positions 238 and 240, and
b) introducing the obtained nucleic acid molecule of step (a) or (b) for expression into a suitable host cell or into a suitable cell extract or cell lysate.

39. The method of claim 38, wherein in step (a) into a nucleic acid molecule encoding the human Interleukin 27 polypeptide or the human Interleukin 27 α-subunit polypeptide or the polypeptide comprising at least 60% sequence identity to the human Interleukin 27 α-subunit polypeptide a nucleotide sequence is introduced mutating the amino acid residue of the α-subunit of human Interleukin 27 at sequence position 238 and/or wherein in step (a) into a nucleic acid molecule encoding the human Interleukin 27 polypeptide or the human Interleukin 27 α-subunit polypeptide or the polypeptide comprising at least 60% sequence identity to the human Interleukin 27 α-subunit polypeptide a nucleotide sequence is introduced mutating the amino acid residue of the α-subunit of human Interleukin 27 at sequence position 240 and/or wherein in step (a) into a nucleic acid molecule encoding the human Interleukin 27 polypeptide or the human Interleukin 27 α-subunit polypeptide or the polypeptide comprising at least 60% sequence identity to the human Interleukin 27 α-subunit polypeptide a nucleotide sequence is introduced mutating the amino acid residues of the α-subunit of human Interleukin 27 at sequence positions 238 and 240 and/or wherein in step (a) into a nucleic acid molecule encoding the human Interleukin 27 polypeptide or the human Interleukin 27 α-subunit polypeptide or the polypeptide comprising at least 60% sequence identity to the human Interleukin 27 α-subunit polypeptide a nucleotide sequence is introduced mutating the amino acid residue of the α-subunit of human Interleukin 27 at sequence position 238 to alanine and/or wherein in step (a) into a nucleic acid molecule encoding the human Interleukin 27 polypeptide or the human Interleukin 27 α-subunit polypeptide or the polypeptide comprising at least 60% sequence identity to the human Interleukin 27 α-subunit polypeptide a nucleotide sequence is introduced mutating the amino acid residue of the α-subunit of human Interleukin 27 at sequence position 240 to alanine and/or wherein in step (a) into a nucleic acid molecule encoding the human Interleukin 27 polypeptide or the human Interleukin 27 α-subunit polypeptide or the polypeptide comprising at least 60% sequence identity to the human Interleukin 27 α-subunit polypeptide a nucleotide sequence is introduced mutating the amino acid residues of the α-subunit of human Interleukin 27 at sequence positions 238 and 240 to alanine.

40-44. (canceled)

45. The method of claim 38, wherein in step (a) into a nucleic acid molecule encoding the human Interleukin 27 polypeptide or the human Interleukin 27 α-subunit polypeptide or the polypeptide comprising at least 60% sequence identity to the human Interleukin 27 α-subunit polypeptide a nucleotide sequence is introduced further mutating 1, 2, 3, 4, 5, 6, 7, 8, 9, 10, 11, 12 or 13 times at one or more positions of any one of position 229, 230, 231, 232, 233, 234, 235, 236, 237, 239, 241, 242, or 243.

46. The method of claim 38, wherein in step (a) into a nucleic acid molecule encoding the human Interleukin 27 polypeptide or the human Interleukin 27 α-subunit polypeptide or the polypeptide comprising at least 60% sequence identity to the human Interleukin 27 α-subunit polypeptide a nucleotide sequence is introduced mutating at least the residues at amino acid positions 234 to 238, at least the residues at amino acid positions 234 to 239, at least the residues at amino acid positions 234 to 240, at least the residues at amino acid positions 234 to 241, at least the residues at amino acid positions 234 to 242, or at least the residues at amino acid positions 234 to 243 and/or wherein in step (a) into a nucleic acid molecule encoding the human Interleukin 27 polypeptide or the human Interleukin 27 α-subunit polypeptide or the polypeptide comprising at least 60% sequence identity to the human Interleukin 27 α-subunit polypeptide a nucleotide sequence is introduced mutating the residues at amino acid positions 229 to 243 (SEQ ID NO: 11).

47. (canceled)


SEQUENCE LISTING (12 sequences)

SEQ ID NO: 1 - protein, 243 aa, Artificial sequence
  mutein of human Interleukin 27 alpha-subunit L162 or L162C
  Variant (162)..(162): Xaa = leucine or cysteine
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNXPEEEEEEEEEEEEERKGLLPGALGSALQGPAQVSWPQLLSTYRLLHSLELVLSRAVRELLLLSKAGHSVWPLGFPTLSPQP

SEQ ID NO: 2 - protein, 243 aa, Artificial sequence
  mutein of human Interleukin 27 alpha-subunit S187A
  Variant (162)..(162): Xaa = leucine or cysteine
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNXPEEEEEEEEEEEEERKGLLPGALGAALQGPAQVSWPQLLSTYRLLHSLELVLSRAVRELLLLSKAGHSVWPLGFPTLSPQP

SEQ ID NO: 3 - protein, 243 aa, Artificial sequence
  mutein of human Interleukine 27 alpha-subunit T238A
  Variant (162)..(162): Xaa = leucine or cysteine
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNXPEEEEEEEEEEEEERKGLLPGALGSALQGPAQVSWPQLLSTYRLLHSLELVLSRAVRELLLLSKAGHSVWPLGFPALSPQP

SEQ ID NO: 4 - protein, 243 aa, Artificial sequence
  mutein of human Interleukin 27 alpha-subunit S240A
  Variant (162)..(162): Xaa = leucine or cysteine
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNXPEEEEEEEEEEEEERKGLLPGALGSALQGPAQVSWPQLLSTYRLLHSLELVLSRAVRELLLLSKAGHSVWPLGFPTLAPQP

SEQ ID NO: 5 - protein, 243 aa, Artificial sequence
  mutein of human Interleukin 27 alpha-subunit S187A and T238A
  Variant (162)..(162): Xaa = leucine or cysteine
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNXPEEEEEEEEEEEEERKGLLPGALGAALQGPAQVSWPQLLSTYRLLHSLELVLSRAVRELLLLSKAGHSVWPLGFPALSPQP

SEQ ID NO: 6 - protein, 243 aa, Artificial sequence
  mutein of human Interleukin 27 alpha-subunit S187A and S240A
  Variant (162)..(162): Xaa = leucine or cysteine
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNXPEEEEEEEEEEEEERKGLLPGALGAALQGPAQVSWPQLLSTYRLLHSLELVLSRAVRELLLLSKAGHSVWPLGFPTLAPQP

SEQ ID NO: 7 - protein, 243 aa, Artificial sequence
  mutein of human Interleukin 27 alpha-subunit S187A, T238A and S240A
  Variant (162)..(162): Xaa = leucine or cysteine
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNXPEEEEEEEEEEEEERKGLLPGALGAALQGPAQVSWPQLLSTYRLLHSLELVLSRAVRELLLLSKAGHSVWPLGFPALAPQP

SEQ ID NO: 8 - protein, 243 aa, Artificial sequence
  mutein of human Interleukin 27 alpha-subunit T238A and S240A
  Variant (162)..(162): Xaa = leucine or cysteine
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNXPEEEEEEEEEEEEERKGLLPGALGSALQGPAQVSWPQLLSTYRLLHSLELVLSRAVRELLLLSKAGHSVWPLGFPALAPQP

SEQ ID NO: 9 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 10 - protein, 234 aa, Mus musculus
MGQVTGDLGWRLSLLLLPLLLVQAGSWGFPTDPLSLQELRREFTVSLYLARKLLSEVQGYVHSFAESRLPGVNLDLLPLGYHLPNVSLTFQAWHHLSDSERLCFLATTLRPFPAMLGGLGTQGTWTSSEREQLWAMRLDLRDLHRHLRFQVLAAGFKCSKEEEDKEEEEEEEEEEKKLPLGALGGPNQVSSQVSWPQLLYTYQLLHSLELVLSRAVRDLLLLSLPRRPGSAWDS

SEQ ID NO: 11 - protein, 228 aa, Artificial sequence
  mutein of human Interleukin 27 alpha-subunit deletion mutant
  Variant (162)..(162): Xaa = leucine or cysteine
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNXPEEEEEEEEEEEEERKGLLPGALGSALQGPAQVSWPQLLSTYRLLHSLELVLSRAVRELLLLSKAG

SEQ ID NO: 12 - protein, 229 aa, Homo sapiens; identical to SEQ ID NO: 9